(19) United States
(12) Patent Application Publication
(10) Pub. No.: US 2007/0231917 A1
(43) Pub. Date: Oct. 4, 2007

(54) Apparatus and method for filtration to enhance the detection of peaks

(75) Inventors: Chulso Moon, Lutherville, MD (US); Atsushi Takano, Tachikawa-shi (JP)

Correspondence Address: HOWREY LLP, C/O IP DOCKETING DEPARTMENT, 2941 FAIRVIEW PARK DR, SUITE 200, FALLS CHURCH, VA, 22042-2924, US

(73) Assignees: Cangen Biotechnologies, Inc.; Dai Nippon Printing Co., Ltd.

(21) Appl. No.: 11/391,471

(22) Filed: Mar. 29, 2006

(51) Int. Cl. G06F 19/00; G01N 33/50

(52) U.S. Cl. 436/86; 702/19

(57) ABSTRACT

Filters and methods for enhancing the identification of peaks is disclosed. In particular, the invention encompasses methods using hole array filters for the purpose of purifying biological fluids to be used in generating mass spectra data.

### FIELD OF THE INVENTION

The present invention relates to methods of enhancing the identification of peaks in mass spectra data for use in the early prediction, detection, and response to treatment of diseases in a human.

### BACKGROUND OF THE INVENTION

The health of a cell and of an organism is reflected by the proteins and other molecules that it contains. The detection, identification, and quantification of proteins and other molecules, such as lipids and carbohydrates, may facilitate disease mechanism elucidation, early detection of disease, prediction of disease, and evaluation of treatments.

Recent advances in genomics research have led to the identification of numerous genes associated with various diseases. However, while genomics research can identify genes associated with a genetic predisposition to disease, there is still a need to characterize and identify markers such as proteins that may be present in an individual patient. A “marker” typically refers to a polypeptide or some other molecule that differentiates one biological status from another. Recently developed methods for molecule detection have made it possible to measure a large fraction of these molecules, opening up a range of new, targeted methods for disease detection, prevention, and treatment. To effectively practice such methods requires the ability to identify individual molecules or markers, often at low concentrations, from mixtures of hundreds or thousands of different compounds.

The use of mass spectrometric methods is replacing gels as the method of choice for bioassays. Exemplary mass spectrometric formats include matrix assisted laser desorption/ionization mass spectrometry (MALDI), see, e.g., U.S. Pat. No. 5,118,937 and U.S. Pat. No. 5,045,694, and surface enhanced laser desorption/ionization mass spectrometry (SELDI), see, e.g., U.S. Pat. No. 5,719,060. The great advantage of mass spectrometry over other technologies for global detection and monitoring of subtle changes in cell function is the ability to measure rapidly and inexpensively thousands of elements in a few microliters of biological fluid. For example, disease processes that result from altered genes, such as cancer, produce altered protein products that circulate in the blood as polypeptides and other molecules of varying size. Mass spectrometry allows for the detection of such products and the subsequent diagnosis and analysis of the disease.

Although many mass spectrometric patterns of complex fluids such as serum defy visual analysis, computational approaches have been used to distinguish subtle differences in patterns from affected individuals compared with unaffected individuals. Efforts to improve the sensitivity of assays have resulted in the application of a number of mass spectrometric formats to the analysis of samples of biological relevance. In addition to the innovations in mass spectrometric techniques, substrates that adsorb an analyte (“chips”) have also been developed and the early designs have been improved upon. However, these methods have thus far proven insufficient to improve the sensitivity of mass spectrometric assays to acceptable levels.

Thus, there exists a need for methods of improving the sensitivity of mass spectrometric assays as they are used in methods of early disease diagnosis, disease prediction, monitoring disease progression or response to treatment, and in identifying which patients are most likely to benefit from particular treatments.

### BRIEF DESCRIPTION OF THE DRAWINGS

FIG. 1 shows an overall flow chart of the invention.

FIG. 2 shows the chromatograms of pre-filtered sera vs. post-filtered sera of both normal & disease A patients.

FIG. 3a shows a cross section of a filter for use in the present invention.

FIG. 3b shows a top view of a hole array of the filter.

FIGS. 4a-4g show the steps that may used in making filters in accordance with the instant invention.

FIG. 5 shows chromatograms of pre-filtered sera vs. post-filtered sera showing the enhancement of a peak in a chemosensitivity screening assay.

FIG. 6a shows the chromatograms of pre-filtered sera vs. post-filtered sera of disease A patients.

FIG. 6b shows the chromatograms of pre-filtered sera vs. post-filtered sera of normal patients.

FIG. 7 shows the chromatograms of pre-filtered sera vs. post-filtered sera of both normal and disease B patients.

FIG. 8 shows the chromatograms of pre-filtered urine vs. post-filtered urine of both normal and disease C patients.

FIG. 9 shows steps that may be used in making filters in accordance with the instant invention.

### SUMMARY OF THE INVENTION

One embodiment of the invention encompasses methods of enhancing peak detection in mass spectrometry.

Another embodiment of the invention encompasses methods of improving the sensitivity and specificity in disease prediction.

Yet another embodiment of the invention encompasses methods of improving the sensitivity and specificity in determining the response to treatment of a disease.

A further embodiment of the invention encompasses a method of increasing sensitivity and specificity in disease detection comprising using filters for the purification of biological fluids; generating mass spectra data from fluid samples from a population with a disease after filtration; generating mass spectra data from fluid samples from a population without disease after filtration; and comparing the two sets of data, wherein the presence of a peak in the biological fluid of a population without disease indicates that said peak is a disease negative marker.

Another embodiment of the present invention encompasses hole array filters.

A further embodiment of the present invention encompasses filters for enhancing the identification of peaks in MALDI-TOF-MS.

### DETAILED DESCRIPTION OF THE INVENTION

A method for enhancing mass spectra data is described. For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented with other compositions and methods, and that any such variation would be within such modifications that do not part from the scope of the present invention. Before explaining the disclosed embodiments of the present invention in detail, it is to be understood that the invention is not limited in its application to the details of any particular embodiment shown, since of course the invention is capable of other embodiments. The terminology used herein is for the purpose of description and not of limitation. Further, although certain methods are described with reference to certain steps that are presented herein in a certain order, in many instances, these steps may be performed in any order as may be appreciated by one skilled in the art, and the methods are not limited to the particular arrangement of steps disclosed herein.

The needs identified in the foregoing Background, and other needs and objects that will become apparent from the following description, are achieved in the present invention, which comprises, but is not limited to, methods for predicting and detecting diseases, and methods for predicting response to the treatment of diseases. The present invention is especially effective for predicting, detecting, and predicting the response to the treatment of diseases such as lung cancer and bladder cancer, but is in no way limited to those diseases. This is because one of the principles embraced by invention relates to the removal of unwanted substances in the samples which results in better peak generation and cleaner data. As such, the filters and methods of the instant invention are not disease specific.

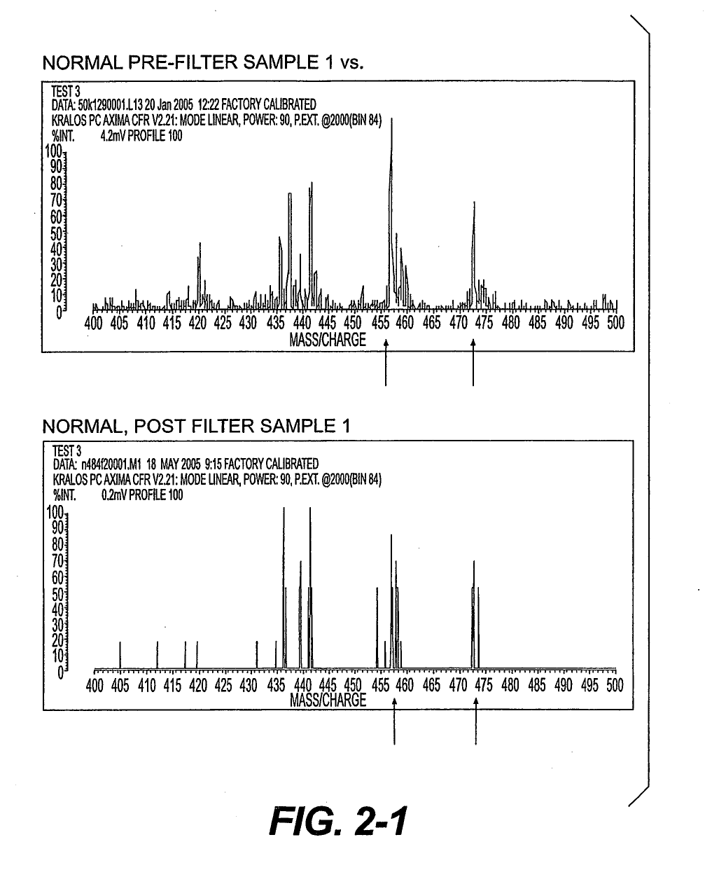

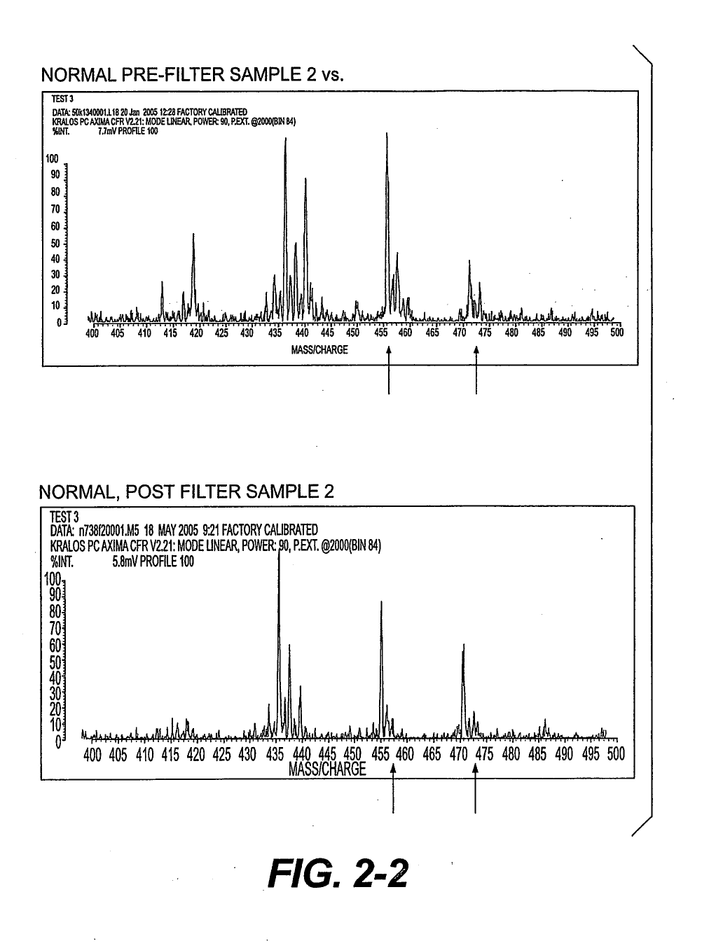

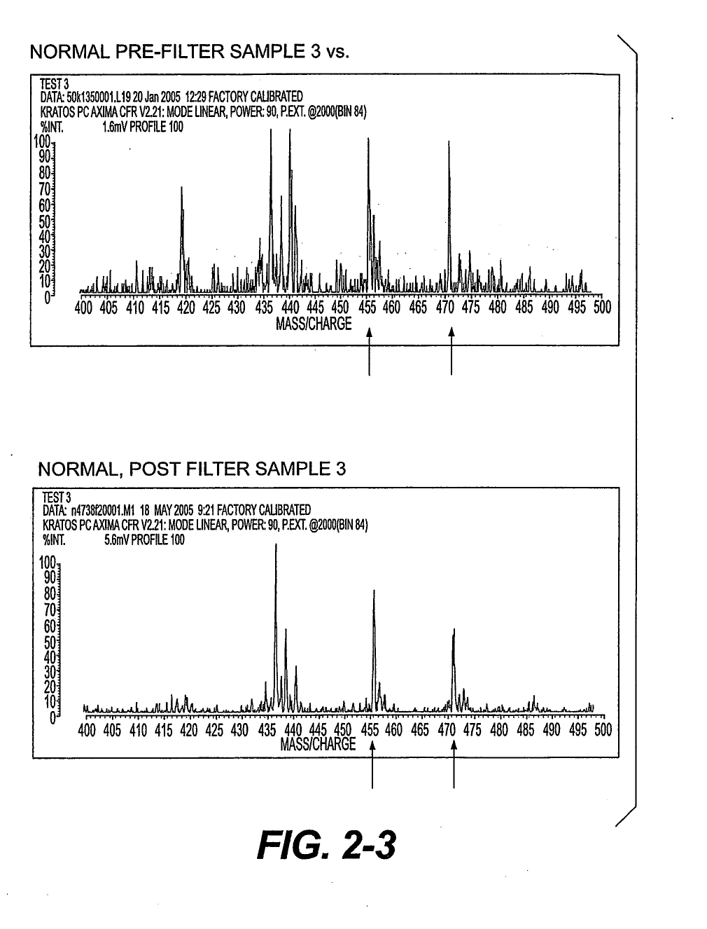

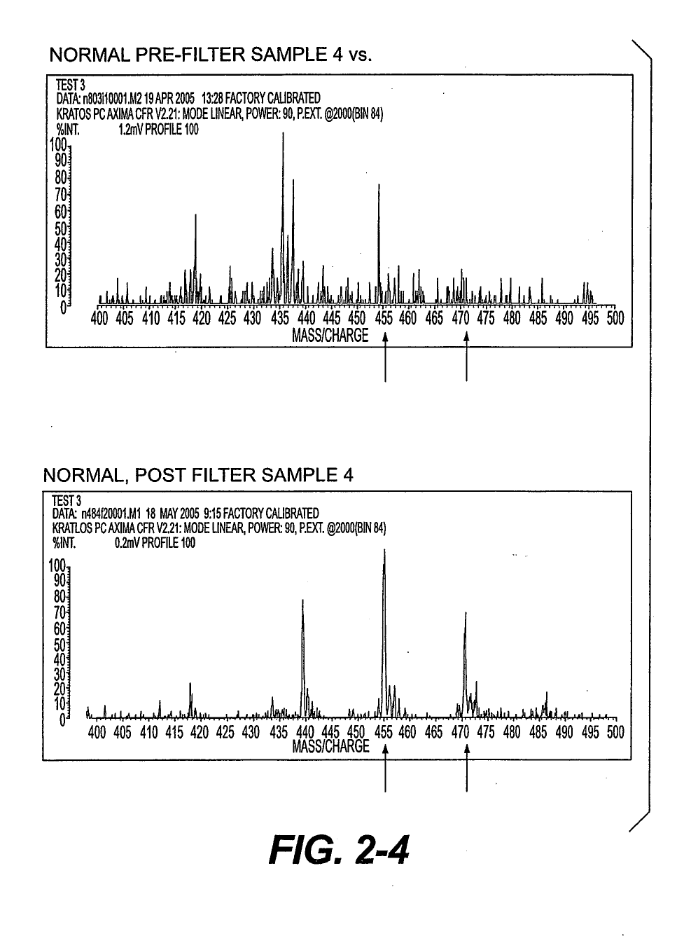

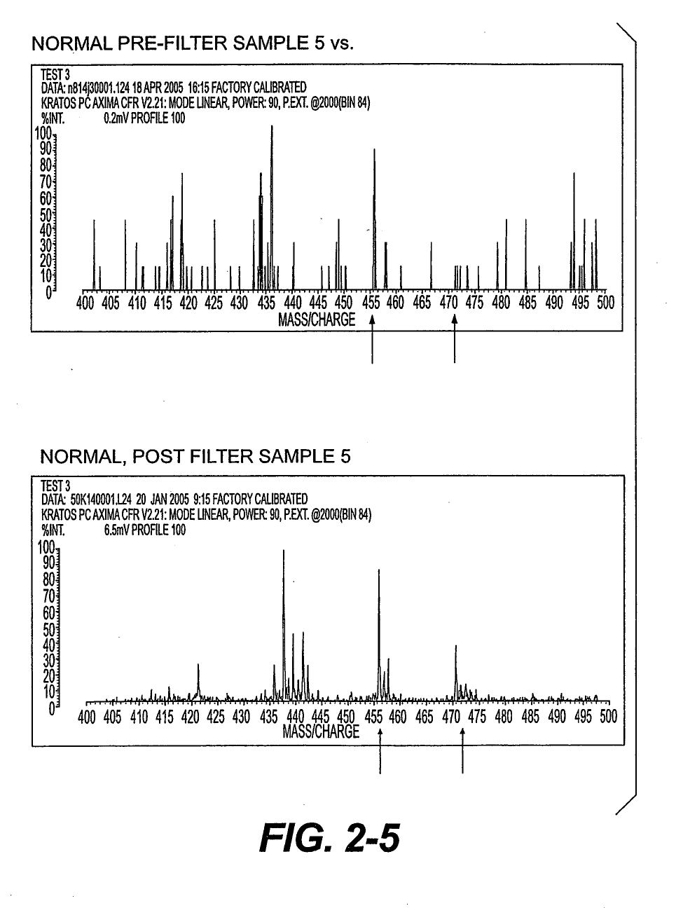

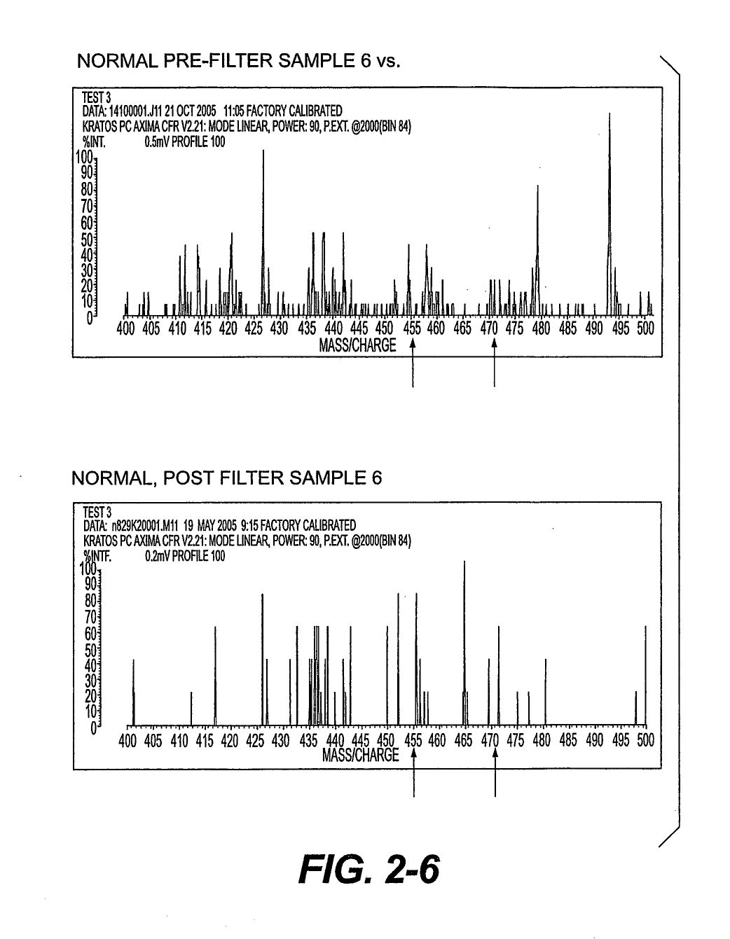

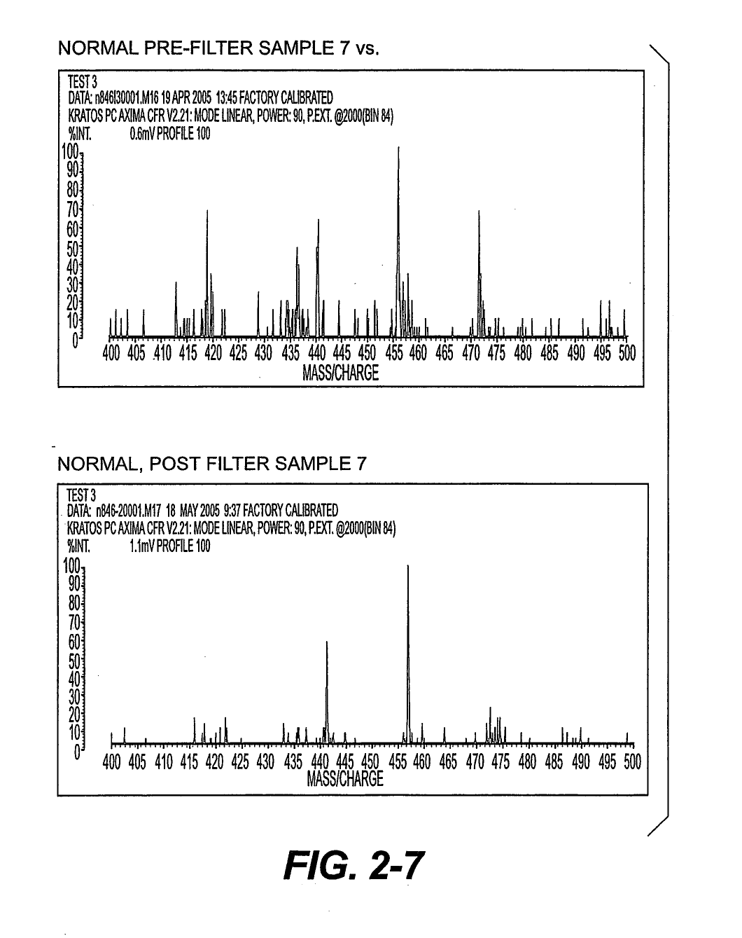

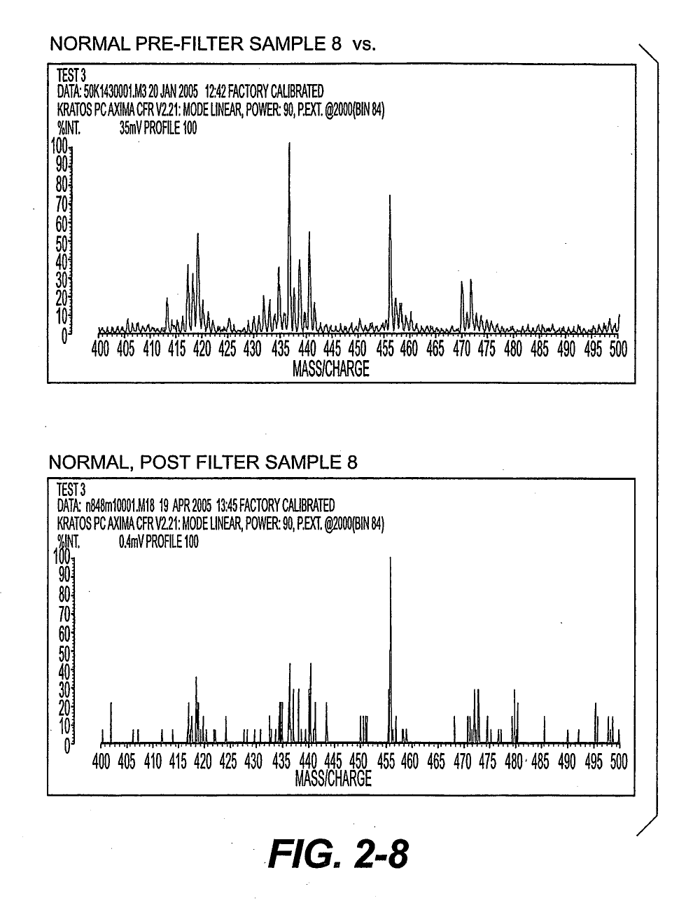

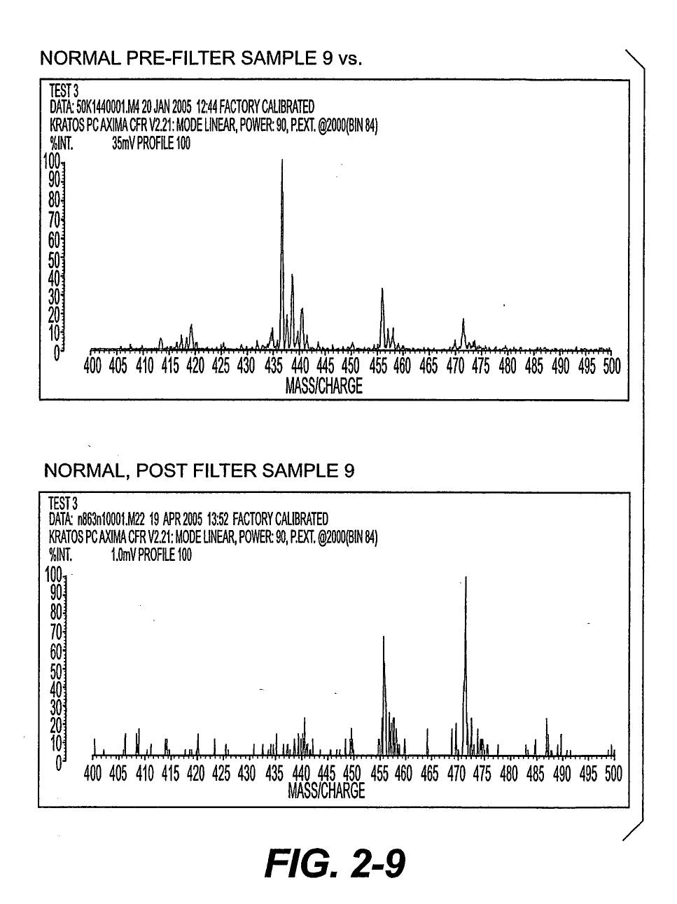

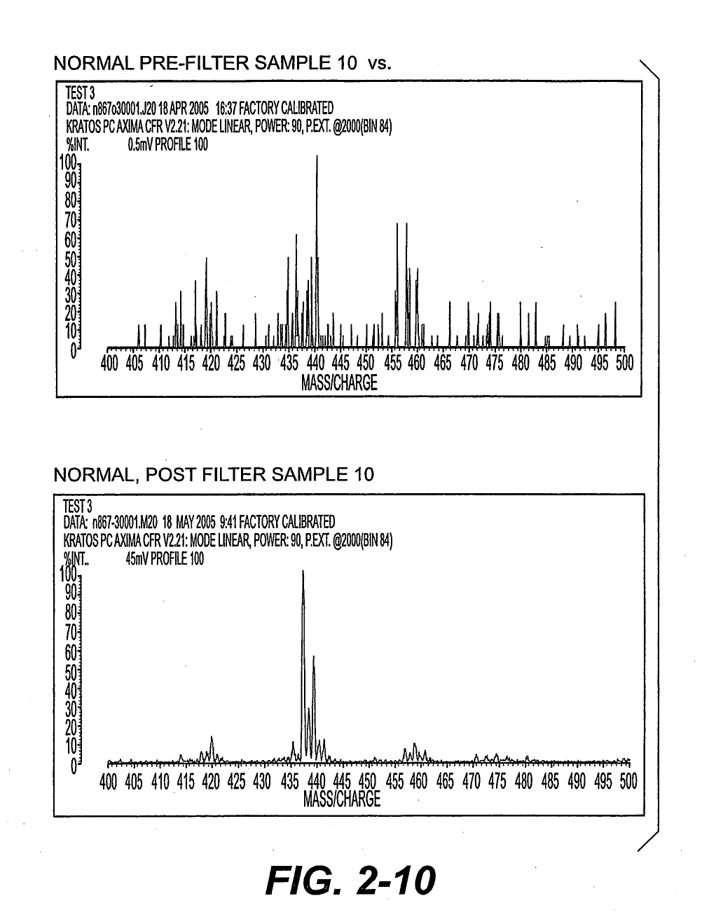

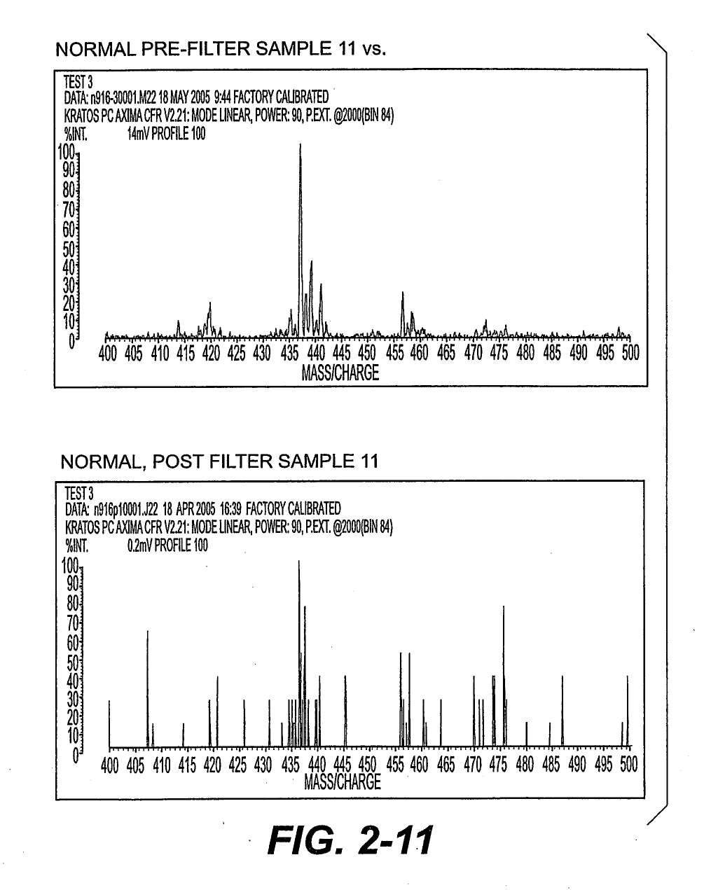

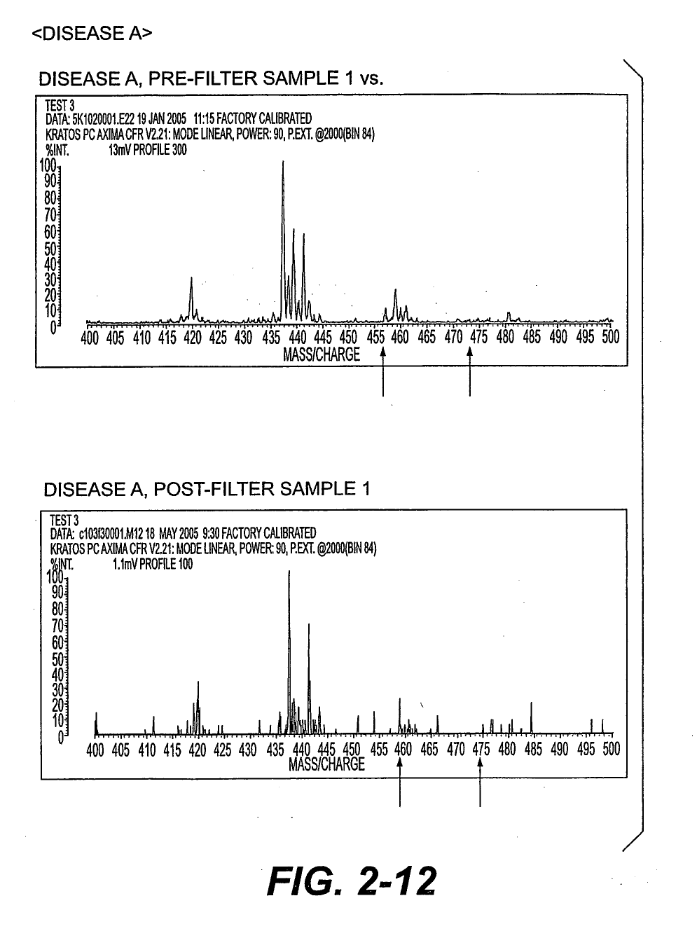

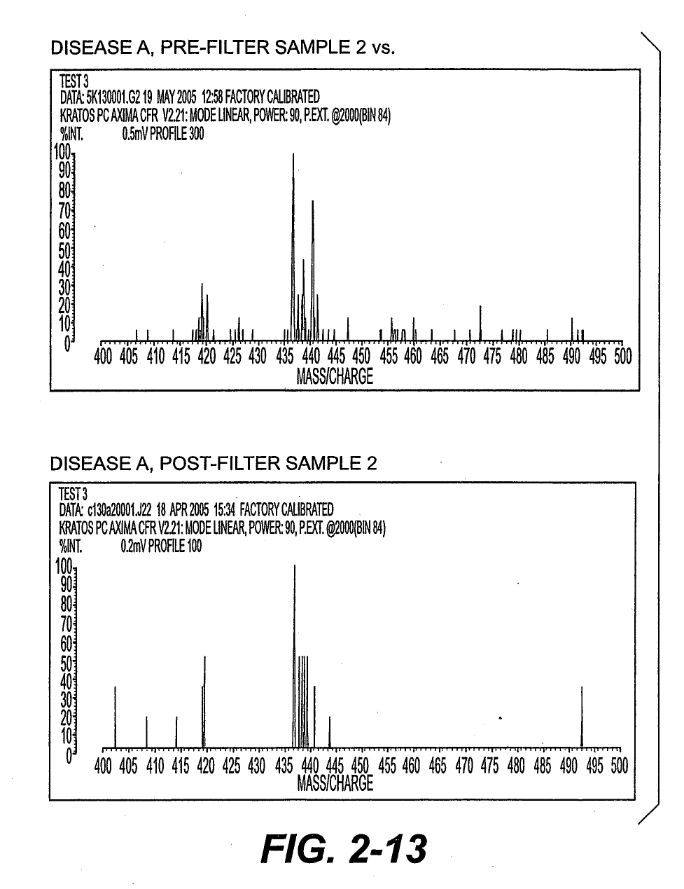

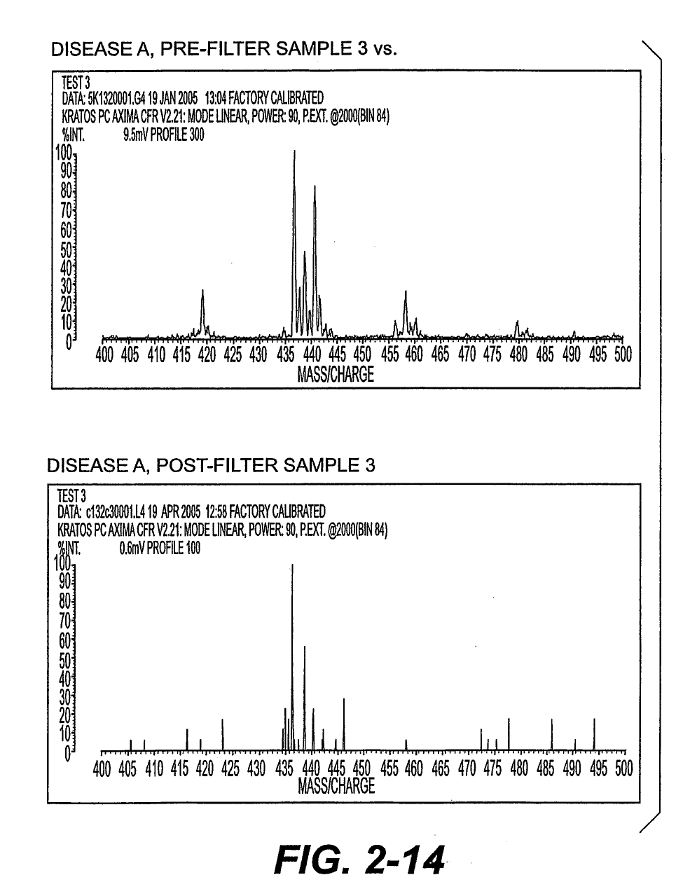

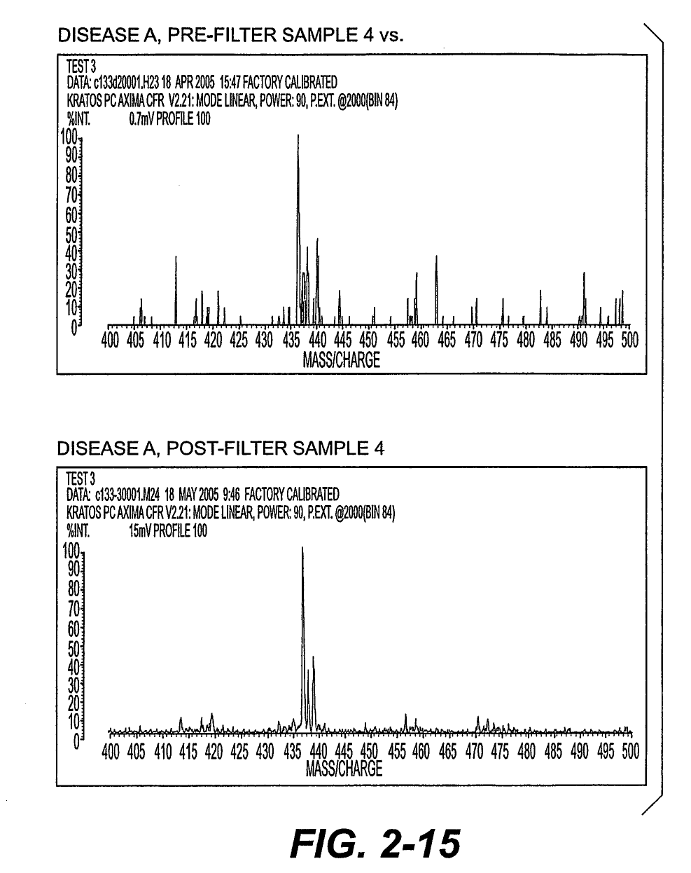

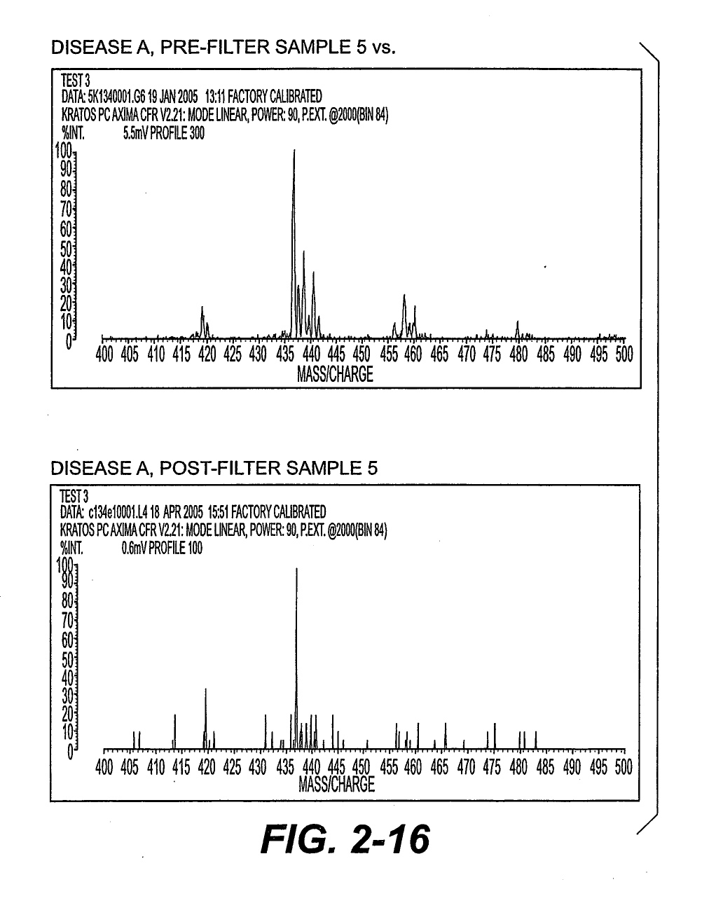

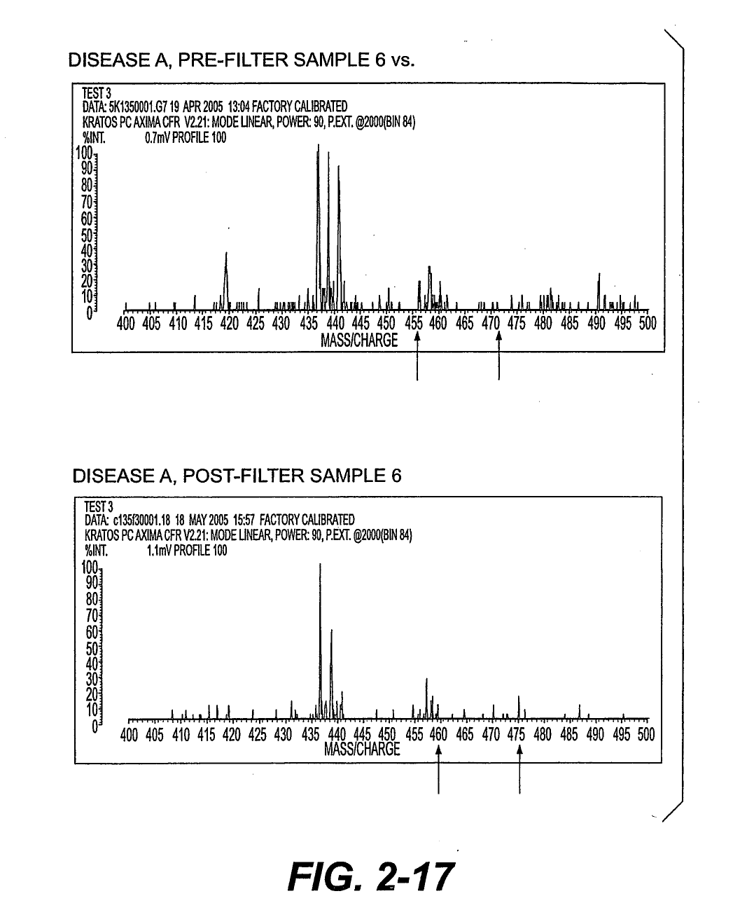

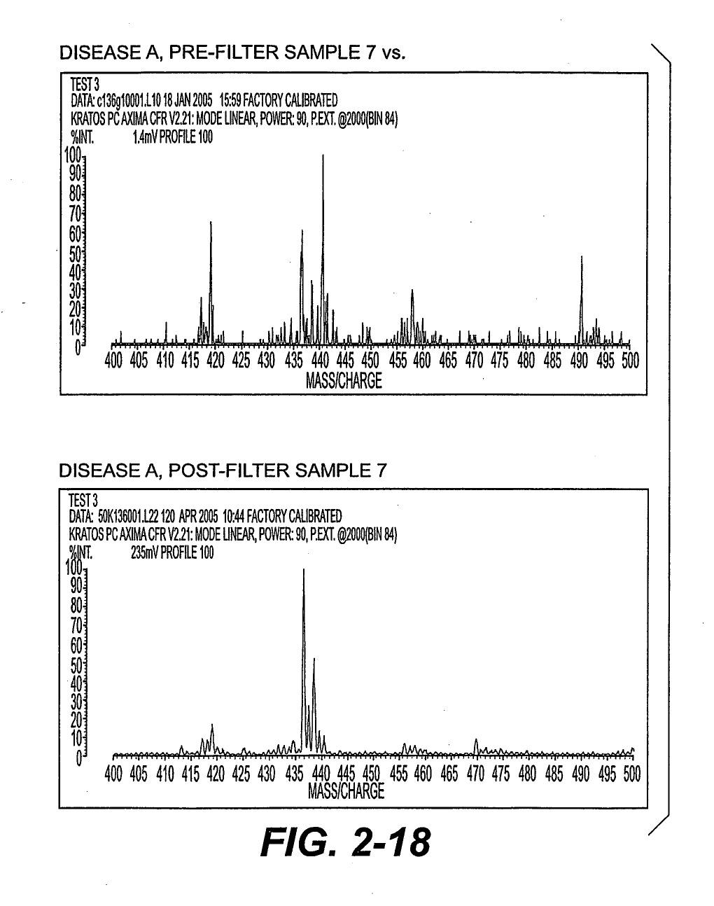

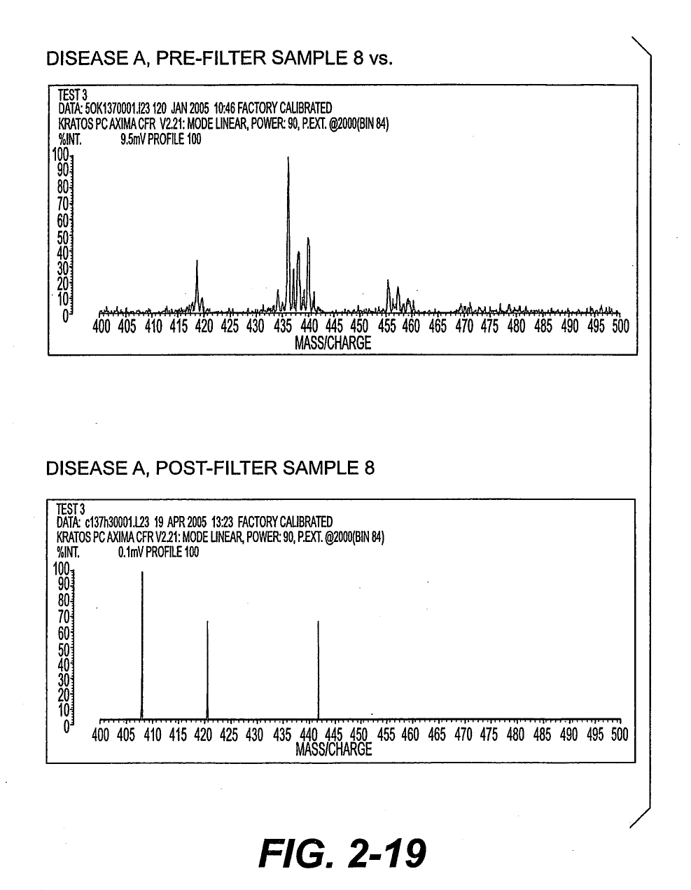

Mass spectroscopic chromatograms were first compared to find differences between normal fluid and fluid of humans with a certain disease, identified herein as disease A. The compared chromatograms focused on a high molecular range because the differences were thought to be not in small molecules but proteins. However, a special peak difference in the high molecular range could not be identified. Accordingly, attempts were made to find differences between two fluid in a low molecular range. Substantial differences were identified at the spots labeled as peaks A and B in FIG. 2 between normal and disease A fluid chromatograms. Normal serum chromatograms show high peaks at the spots corresponding to A and B, but disease A fluid chromatograms do not show any peak or show only small peaks at those spots.

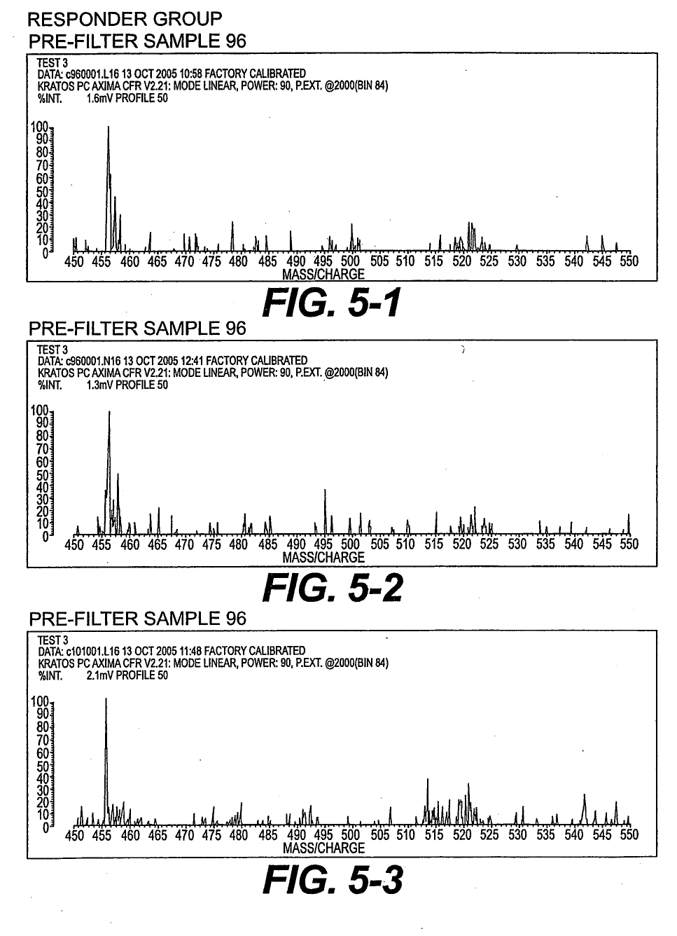

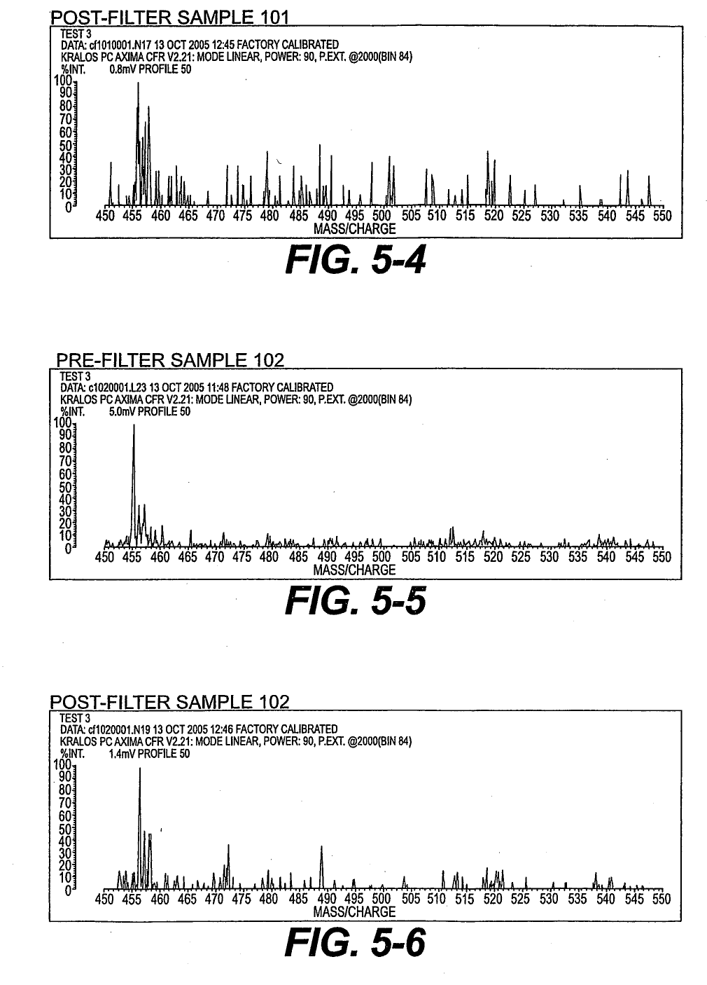

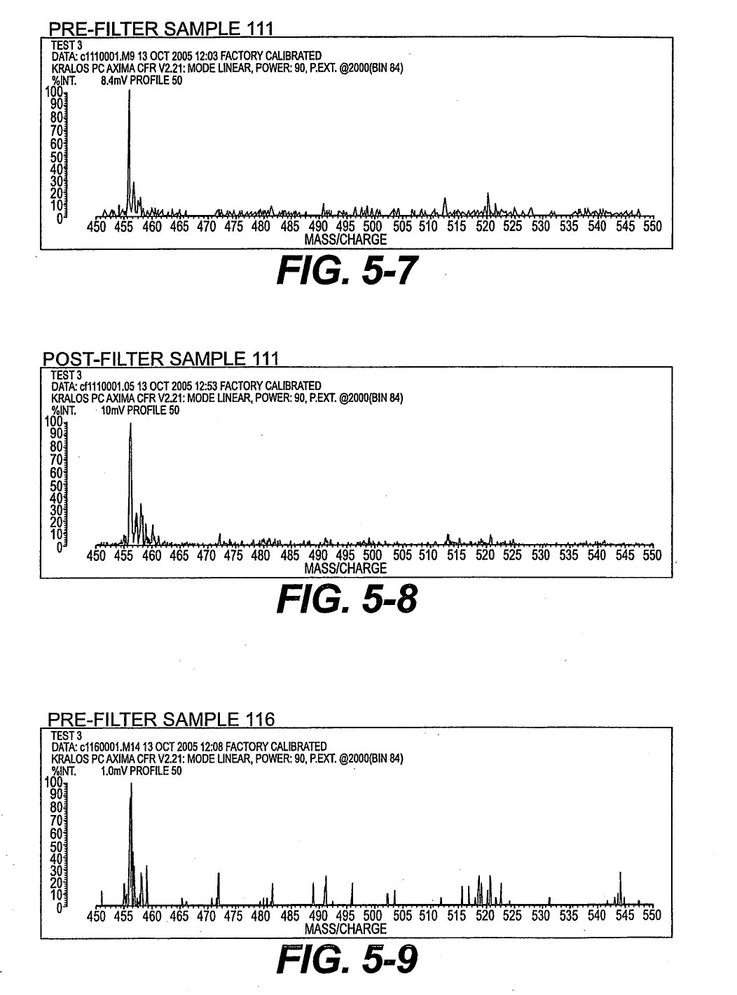

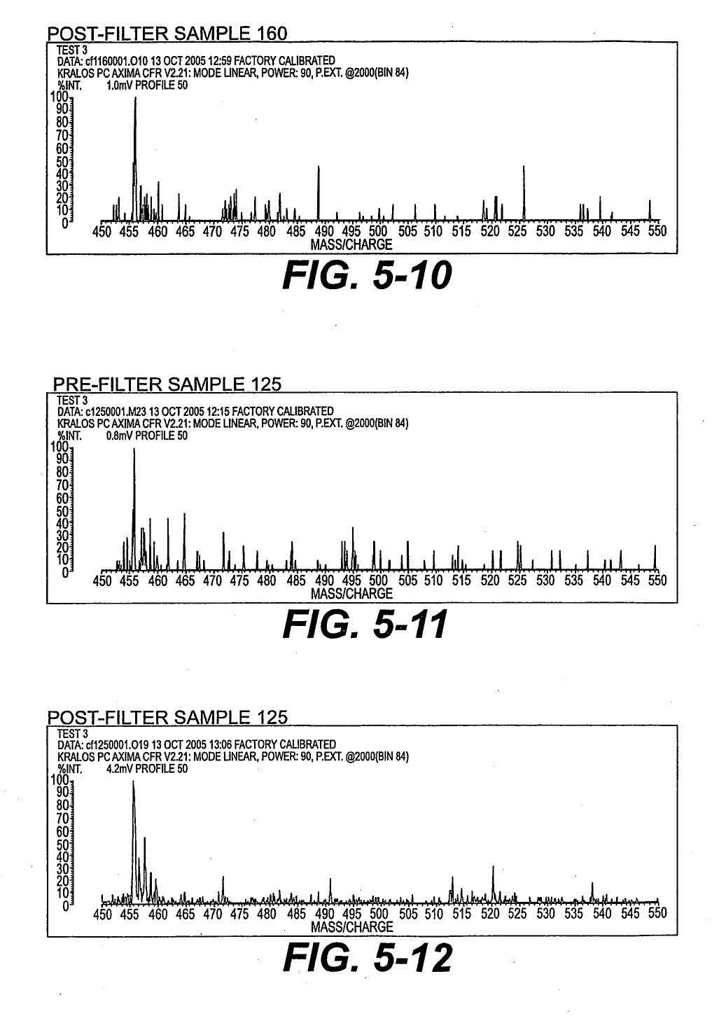

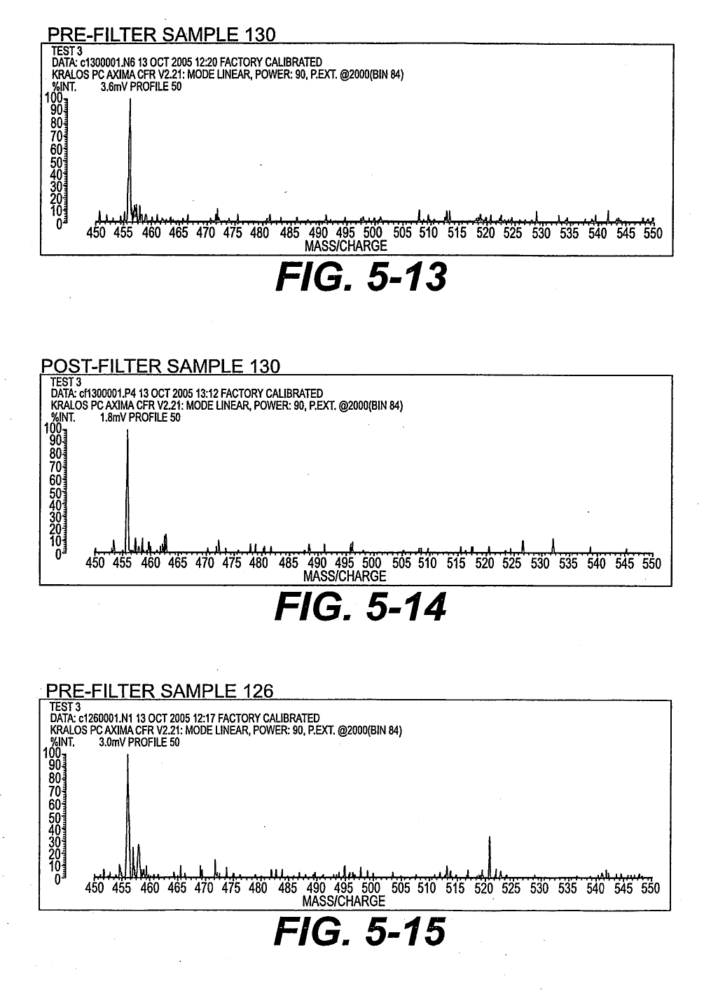

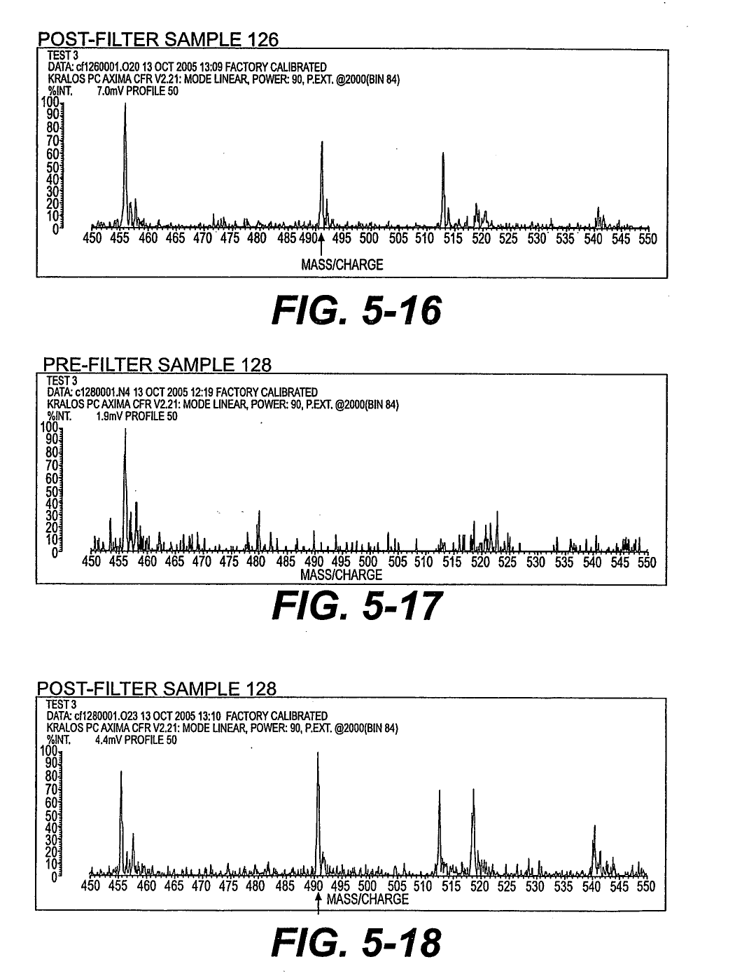

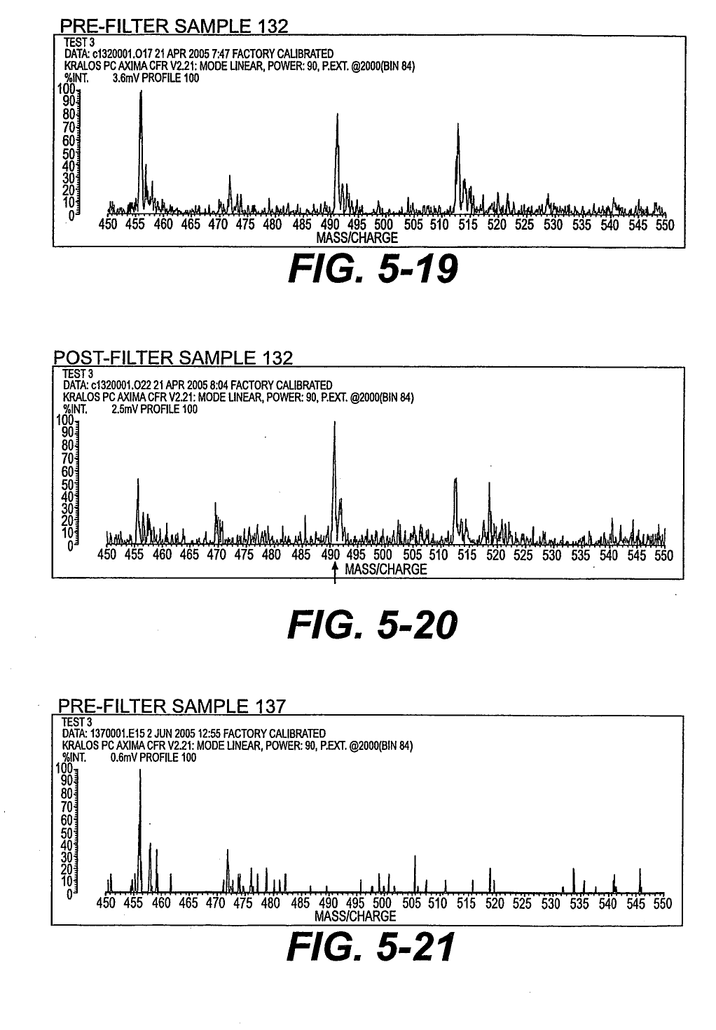

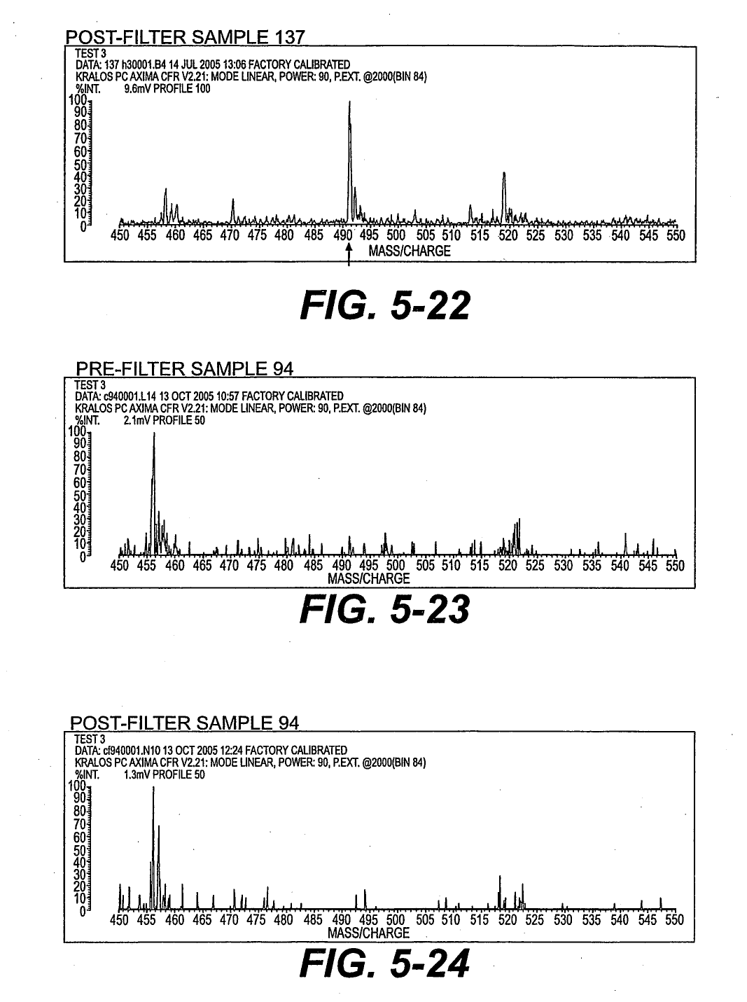

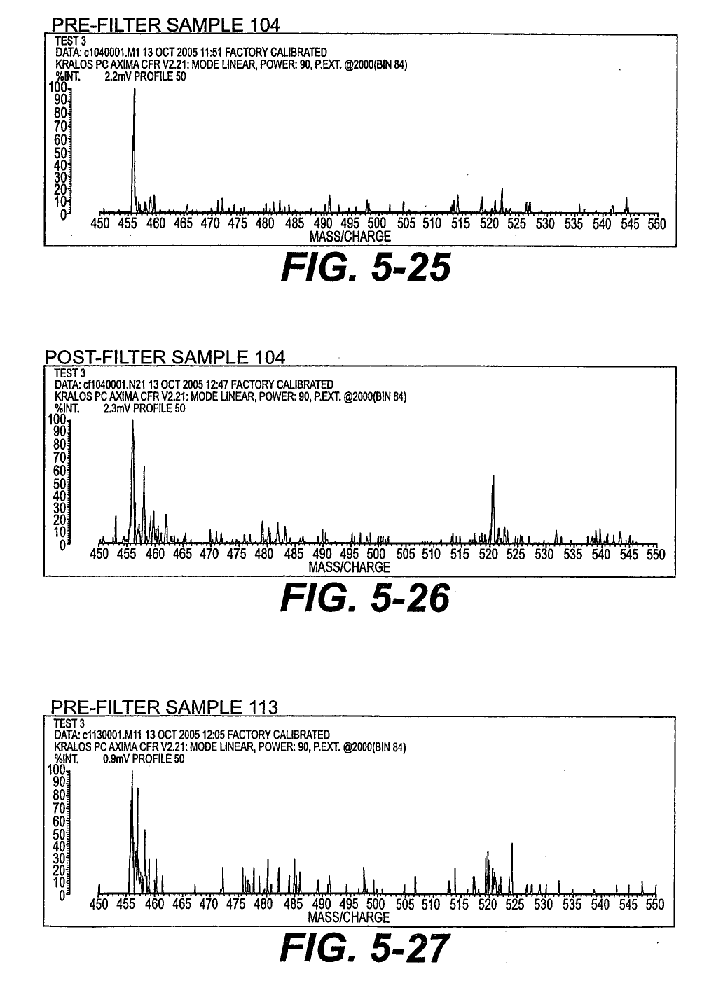

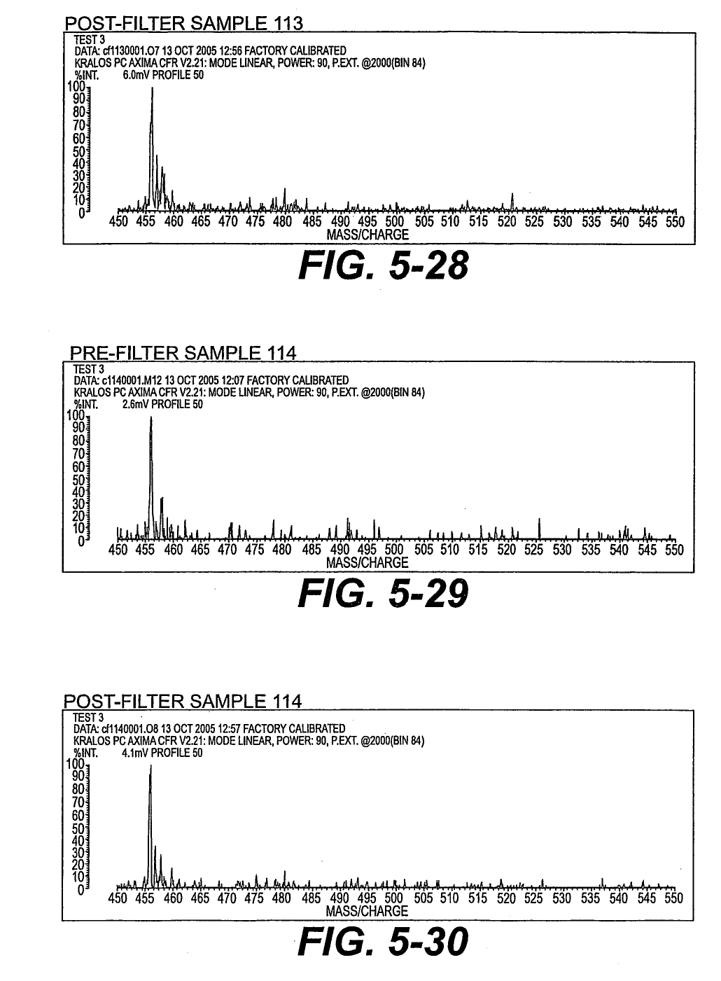

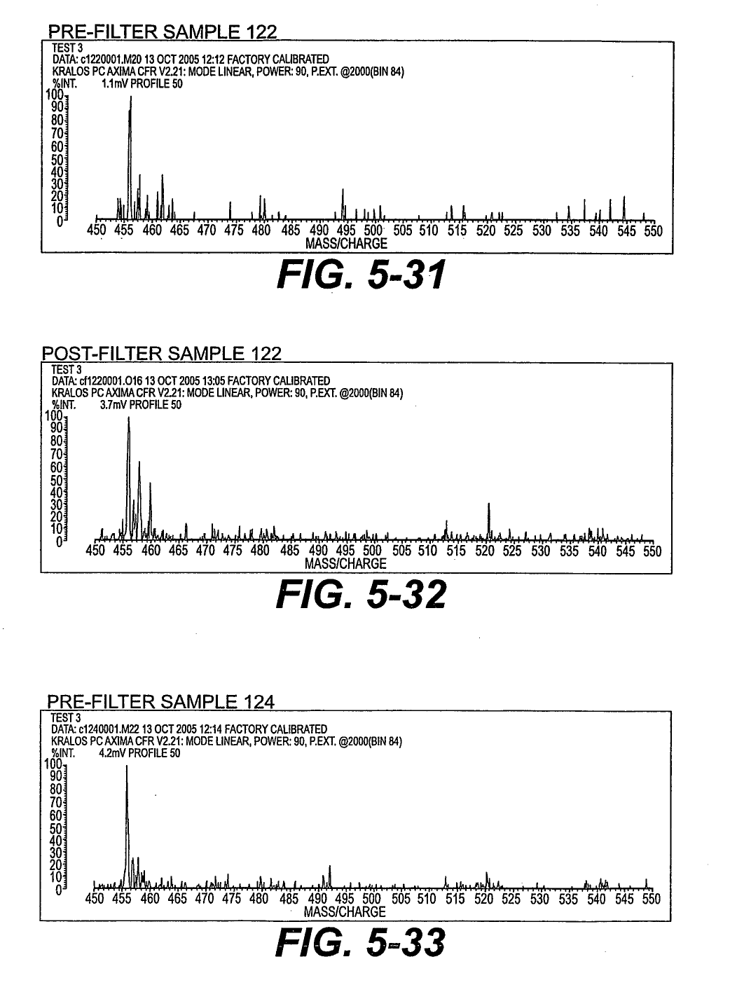

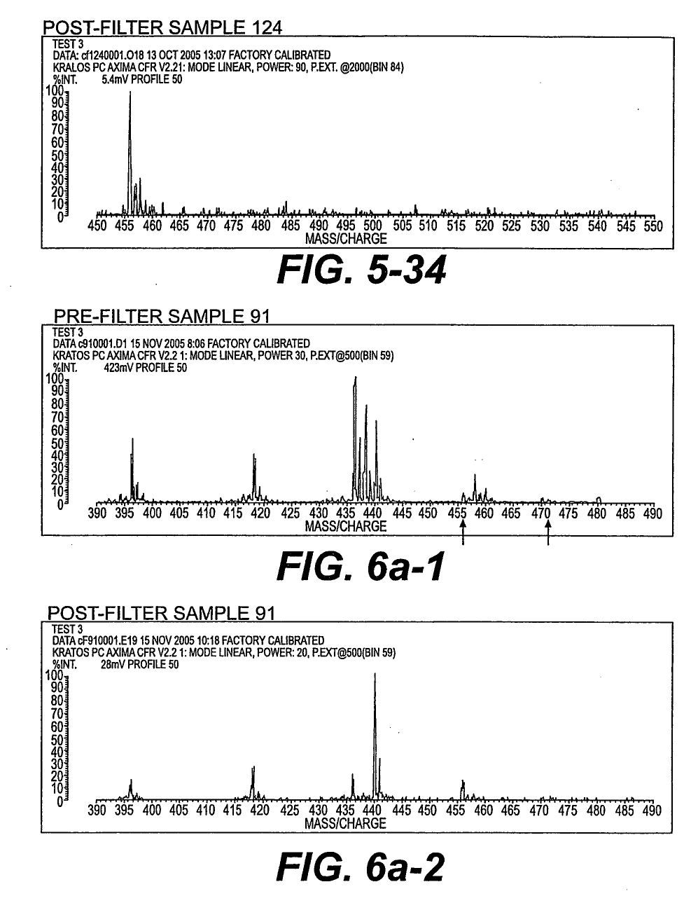

In another aspect of the invention, mass spectroscopic chromatograms were compared between groups responding to a particular chemotherapy treatment with those that did not respond to that particular chemotherapy treatment. A peak was identified in a substantial number of the non-responders. These results can be seen in FIG. 5.

While the data generated by the above assays proved useful, it was determined that the data could be improved. Surprisingly, it was determined that a purification step of biological fluid enhances the ability to detect the presence or absence of peaks indicating biomarkers. Hole array filters were used to purify the serum. As a result of the filtration, sensitivity increased by 10% and specificity increased by 25%.

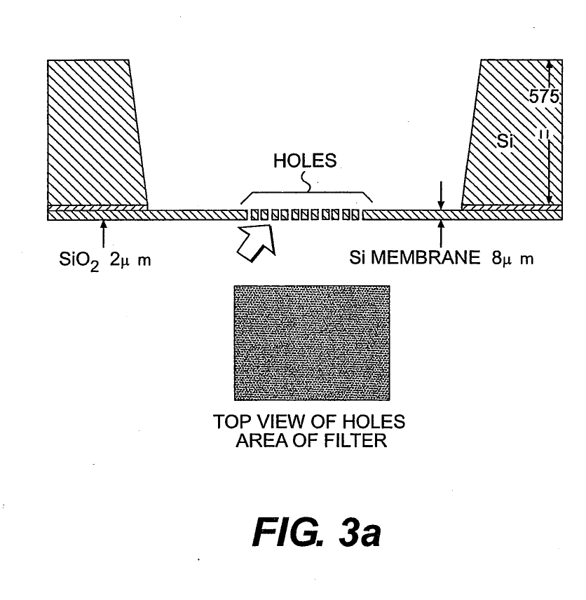

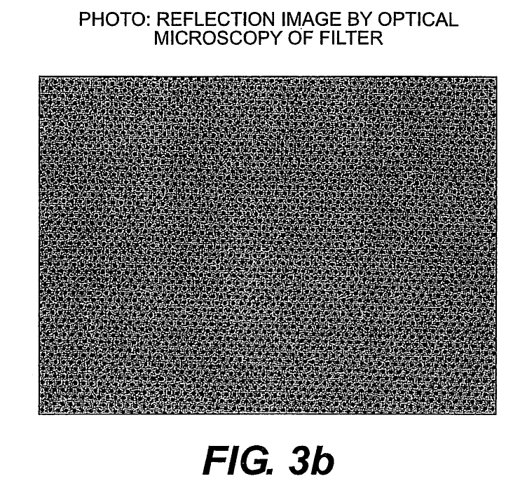

The filters for use in the present invention may comprise an array of holes formed in a silicon membrane of about 3 to 20 μm in thickness. Preferably, the membrane thickness is between about 6-10 μm. If the thickness is less than 6 μm, the hole array area becomes very fragile. If the thickness is more than 10 μm, filtration time becomes increases due to the resistance of the hole surface area A thickness of more than 10 μm also increases the difficulty of making smooth holes. The size of the hole array may be between 1 mm by 1 mm and 10 mm by 10 mm. If the area is smaller than 1 mm by 1 mm, the amount of filtered biological fluid is not enough to generate adequate data. If the area is larger than 10 mm by 10 mm, the amount of biological fluid becomes too much and the filter becomes more expensive. The size of the holes in the array may be from about 2-20 μm and preferably about 1-10 μm. In this instance, the term size refers to diameter for a circular hole and diagonal for a square hole. If the size is smaller than 1 μm, the filter hole array area tends to break when negative pressure is applied. If the size is larger than 10 μm, unwanted compounds of biological fluid tend to go through the filter holes and the filtration process becomes insufficient. The hole pitch, or distance between holes, may be about three times the size of the holes (preferably 3-30 μm) but may be more or less than three times the hole size depending on the particular application (see FIGS. 3a and 3b). Hole array filters consist of mainly two areas. A thin area with a hole array and a thick outer area to improve filter rigidity. The material of the filter is rigid and easily processed in to precise designed patterns. Filter materials include, but are not limited to, materials such as metal or semiconductor material. One example is Si(thick layer)/SiO2/Si(thin layer with hole array). If Si(thick layer) is tapered toward Si(thin layer with hole array), the flow of biological fluid through the hole array filter becomes smoother. Another material that may be used for hole array filters is Ni/Cu. The hole array filter material should be rigid and with evenly made holes matching the designed size.

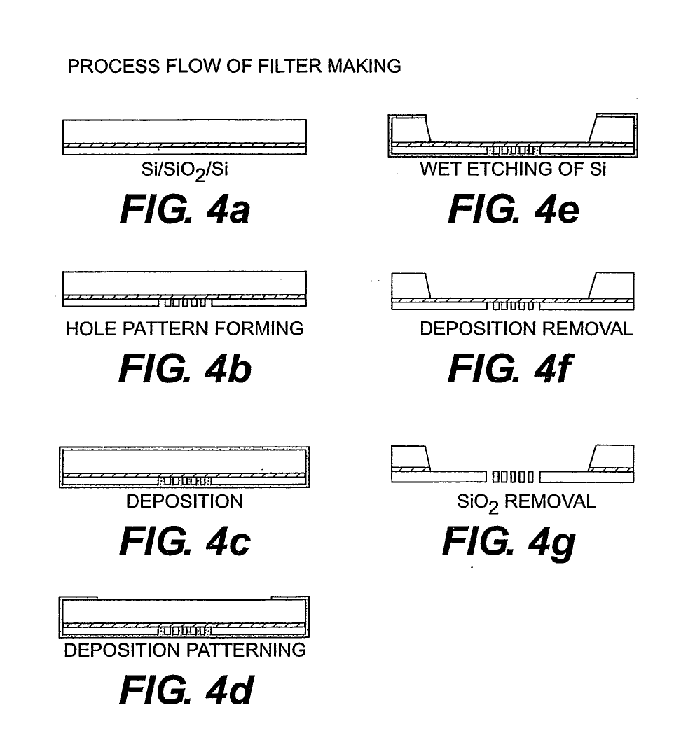

The filters used in the present invention may be made by any method known in the art of lithography or filter making. In one exemplary method, a silicon substrate of about 575 μm thickness may be used as the starting material. A thin layer of silicon dioxide may then be formed on one side of the substrate using any common method such as chemical vapor deposition (CVD) or through further oxidation of the surface portion of the substrate by exposure to an oxygen containing plasma. The silicon dioxide layer may be about 2 μm thick. A thin layer of silicon may be formed on the oxide layer by any method such as CVD or thin film crystallization (see FIG. 4a). The substrate may then be flipped over so the thin crystalline silicon film is on the bottom or backside of the substrate. This silicon naturally develops a very thin layer of silicon dioxide of a thickness on the order of a few Angstroms. This substrate is typically called Silicon On Isolator (SOI) substrate.

The resist material is coated on the SOI surface. Resist material can be photoresist for photo exposure such as Ultra Violet light and electron beam resist for electron beam exposure at the following processes. Then patterned mask is applied onto or in proximity to the resist. Then ionizing radiation such as ultra violet light or electron beam is applied to the resist through the patterned mask. After the mask is removed, unnecessary pattern portion of the resist is removed by removing material such as solvent. Then Si layer is etched either dry or wet process to make a certain shaped hole array as shown in FIG. 4b. after removing the whole resist. In such cases, silicon dioxide layer works as etching stopping layer.

A protective layer may then be applied over the entire substrate including over the hole array (FIG. 4c). A portion of the protective layer on the top side of the substrate and symmetrically arranged compared to the underlying hole array but wider than the hole may then be removed through a mask and resist etching process (FIG. 4d). A wet etch of the exposed substrate may then be performed until the oxide layer is reached resulting in the exposure of the oxide layer surrounding the underlying hole array and tapered walls of the side of the exposed silicon substrate, as shown in FIG. 4e. The remainder of the protective layer may then be removed by a wet or dry etching process as shown in FIG. 4f. The exposed portion of the oxide layer may then be removed by a wet or dry etching process resulting in a finished filter as shown in FIG. 4g.

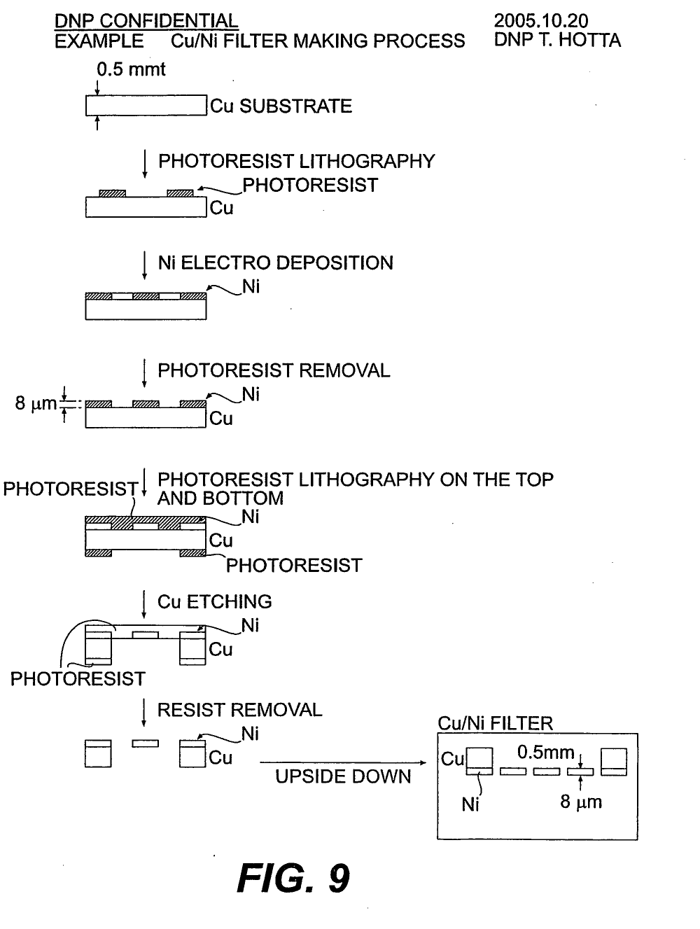

Filters in accordance with the instant invention may also be made with other materials such as Ni/Cu. The steps are similar to those above and are shown in FIG. 9.

Although specific steps and processes have been used to describe the formation of the filters used in the present invention, these steps and processes are exemplary only. As is well known in the art, any processes may be used to form a hole array in a thin layer of silicon. Additionally, the thicknesses of the different layers and sizes of the holes and distances between the holes are provided as exemplary only and are not meant to be limiting in any manner. Additionally, the word “hole” is not meant to be limited to a void of any particular shape but may be round, square, triangle, or any other shape. As such, cross sectioning of the holes need not be cylindrical in shape. Further, the filter material is not limited to silicon as the filter may comprise any common filter material.

It should also be noted that any suitable biological samples may be used in embodiments of the invention. Biological samples include tissue (e.g., from biopsies), blood, serum, plasma, nipple aspirate, urine, tears, saliva, cells, soft and hard tissues, organs, semen, feces, urine, and the like. The biological samples may be obtained from any suitable organism including eukaryotic, prokaryotic, or viral organisms.

The biological samples may include biological molecules including macromolecules such as polypeptides, proteins, nucleic acids, enzymes, DNA, RNA, polynucleotides, oligonucleotides, nucleic acids, carbohydrates, oligosaccharides, polysaccharides; fragments of biological macromolecules set forth above, such as nucleic acid fragments, peptide fragments, and protein fragments; complexes of biological macromolecules set forth above, such as nucleic acid complexes, protein-DNA complexes, receptor-ligand complexes, enzyme-substrate, enzyme inhibitors, peptide complexes, protein complexes, carbohydrate complexes, and polysaccharide complexes; small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphospho-sugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), mitochondrial and chloroplast function inhibitors, electron donors, carriers and acceptors, synthetic substrates for proteases, substrates for phosphatases, substrates for esterases and lipases and protein modification reagents; and synthetic polymers, oligomers, and copolymers. Any suitable mixture or combination of the substances specifically recited above may also be included in the biological samples.

### EXAMPLE 1

Filter Evaluation

In order to more fully optimize and characterize the present filtration methods, the hole array filters identified in the following table were evaluated for their ability to cleanse sample and thereby improve the sensitivity and specificity of the present methods.

(unit: μm)

| Filter Name | Designed hole diameter | Designed pitch |
|---|---|---|
| 1-11P | 1 | 11 |
| 5-10P | 5 | 10 |
| 5-20P | 5 | 20 |
| 5-55P | 5 | 55 |
| 10-40P | 10 | 40 |
| 10-110P | 10 | 110 |

Samples were filtered with each of the above filters. Evaluation by MALDI-TOF-MS found the following trend in cleansing effect: 1-11P>5-10P>5-20P>5-55P>10-40P>10-110P. That is, filter 1-11P had the greatest cleansing effect for the samples tested. However, each progressive level of filtration increased the ability to identify certain peaks as compared to the prior level of filtration.

Hole array filters as described in the following table were also evaluated for cleansing effect:

Designed hole diameter
2 μm
3 μm
4 μm
5 μm
6 μm
7 μm
8 μm
9 μm

Some of the 2 μm filters were broken when negative pressure was applied during the filtering process. This is likely due to small holes which cause more pressure in the filter layer, when the filter layer is covered by biological fluid. The remainder of the 2 μm filters worked as expected. The results from the 9 μm hole filters tend to give less filtering effect than the smaller hole filters. It is likely that this is because larger holes let more unwanted substances in the biological fluid through the filter in some cases but still work to filter unwanted substances in other cases. This evaluation indicated that the filter hole size between 2 and 9 μm work well and optimum hole size of filter is approximately 5.5 um (=(2+9)/2). Such filters work well to remove unwanted substances to reduce unwanted substance related peaks in the data.

### EXAMPLE 2

Method and Apparatus

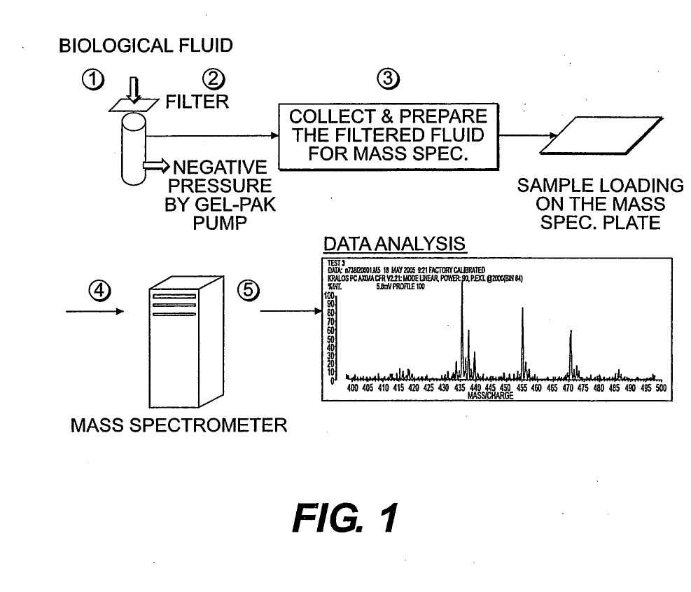

Example embodiments are now described with respect to FIG. 1 and FIG. 2. FIG. 1 is a flow diagram that illustrates an overview of one embodiment of a method for generating a disease A-screening.

Generating Data from Un-Filtered Serum Samples

MALDI-TOF-MS was used to generate a spectra sample data set representing distinct protein/peptide patterns in serum. In one clinical investigation, sera either from patients with disease A or healthy controls were obtained before surgical procedures. All final diagnoses were confirmed by histopathology and all controls were at high risk for disease A, but without evidence of disease A based on clinical presentation and CT scan examination.

The sera were prepared for evaluation by the mass spectrometer by making a matrix of serum samples. The mass spectrometer matrix contained saturated alpha-cyano-4-hydroxycinnamic acid in 50% acetonitrile-0.05% trifluoroacetic acid (TFA). The sera were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Generating Data from Filtered Serum Samples

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1). The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spread out, filling the inner square of filter. The negative air flow was applied by pumping the air pump manually. The dropped sera solutions from the filter to the micro tube were collected and transferred to the new tube. The filtered serum was further diluted 1:100 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Results

Comparison between normal sera data and disease A sera data from FIG. 2 in the pre-filtered condition:

First, normal sera and disease A sera were tested in the pre-filtered condition. 11 normal sera and 8 disease A sera were treated by the same method as described above and the chromatogram profiles were created. When the chromatogram profiles were compared on a fixed range, a substantial difference was found between normal sera and disease A sera in the spots corresponding to points A and B in FIG. 2.

As seen in FIG. 2, the chromatograms of normal sera showed peaks in the spots A and B. But, the chromatograms of disease A sera had substantially no peaks at the spots A and B.

In the case of normal sera as shown in FIG. 2, 10 of 11 samples showed peaks at spot A. 6 samples of 11 samples show peaks in the spot B. However, in the case of disease A sera, as shown in FIG. 2, all 8 samples show no peaks in the spots A and B. The table below summarizes these results:

Spot

| Sera Group | A | B |
|---|---|---|
| No. of samples with | 10/11 | 6/11 |
| peaks in Normal Sera | | |
| No. of samples with | 0/8 | 0/8 |
| peaks in Disease A Sera | | |

Comparison between normal sera data and disease A sera data in the filtered condition:

Referring to FIG. 2, normal sera and disease A sera were treated by the same filtration method as described above. The chromatogram profiles were prepared from the filtered sera. When the chromatogram profiles were compared on a fixed range with normal sera, all 11 samples showed peaks in the spot A and 7 of 11 samples showed peaks in the spot B. However, in case of disease A sera, the result was the same in the pre-filtered condition. All 8 samples show no peaks in the spot A and B. The table below summarizes these results:

Spot

| Sera Group | A | B |
|---|---|---|
| No. of samples with | 11/11 | 7/11 |
| peaks in Normal Sera | | |
| No. of samples with | 0/8 | 0/8 |
| peaks in Disease A Sera | | |

It should be noted, in the case of disease A sera, low peaks in the spots of A and B were reduced or eliminated after filtering the sera. In other words, the use of the filter accentuated the differences in the mass spectrographs of sera from people with disease A compared to sera from people not suffering from disease A. This enhanced the detection of the peaks.

Filtering Effect

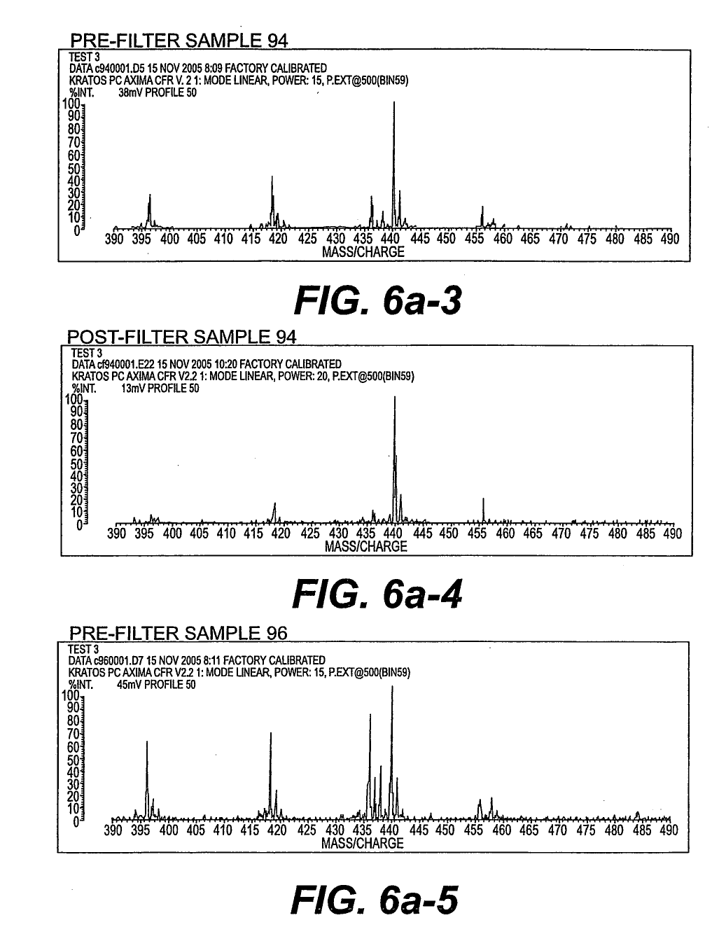

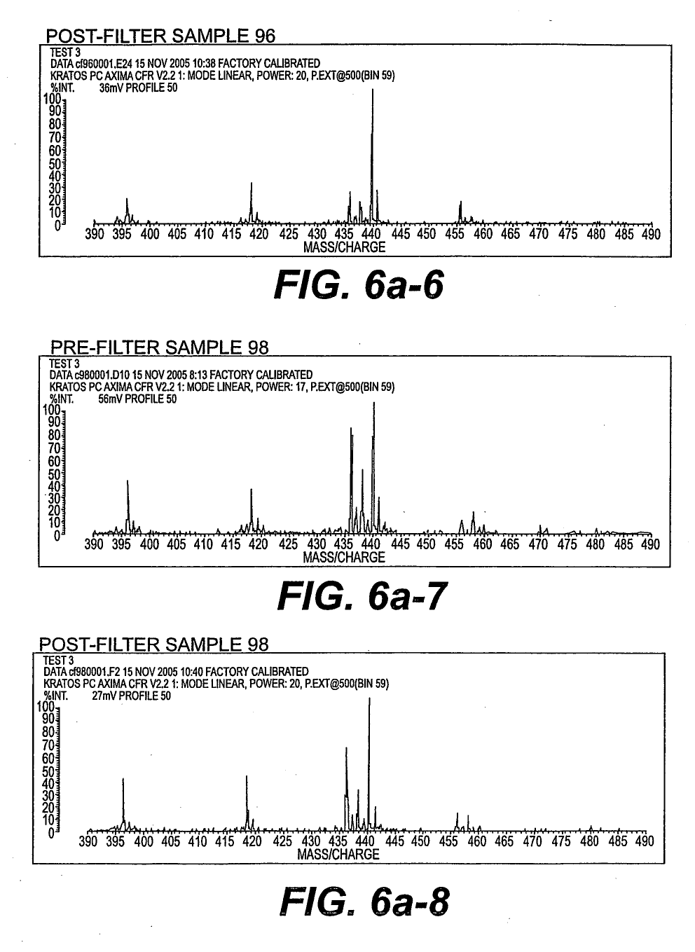

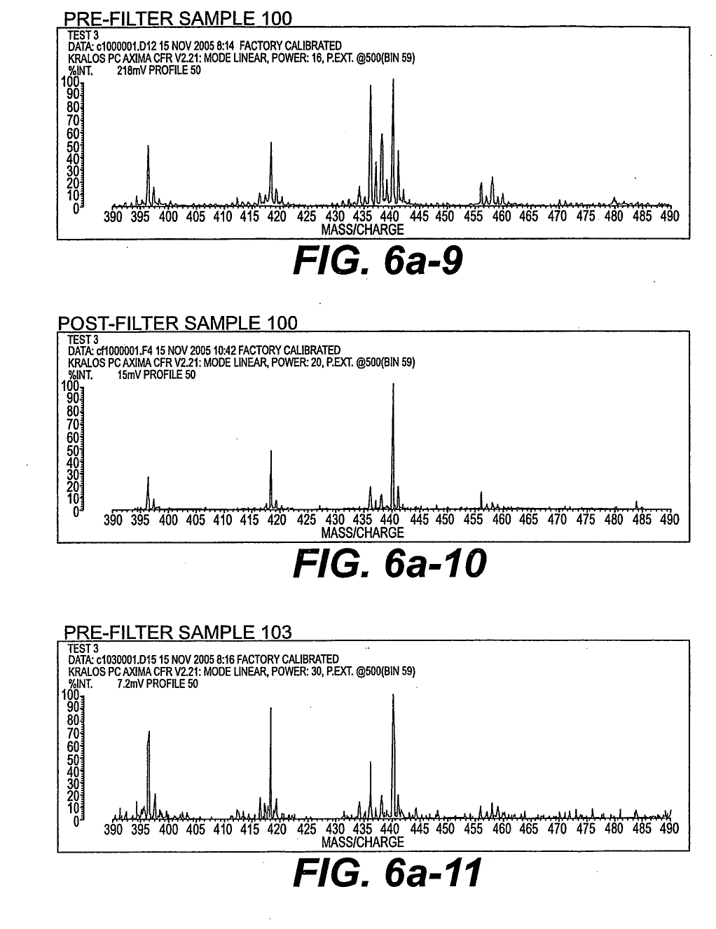

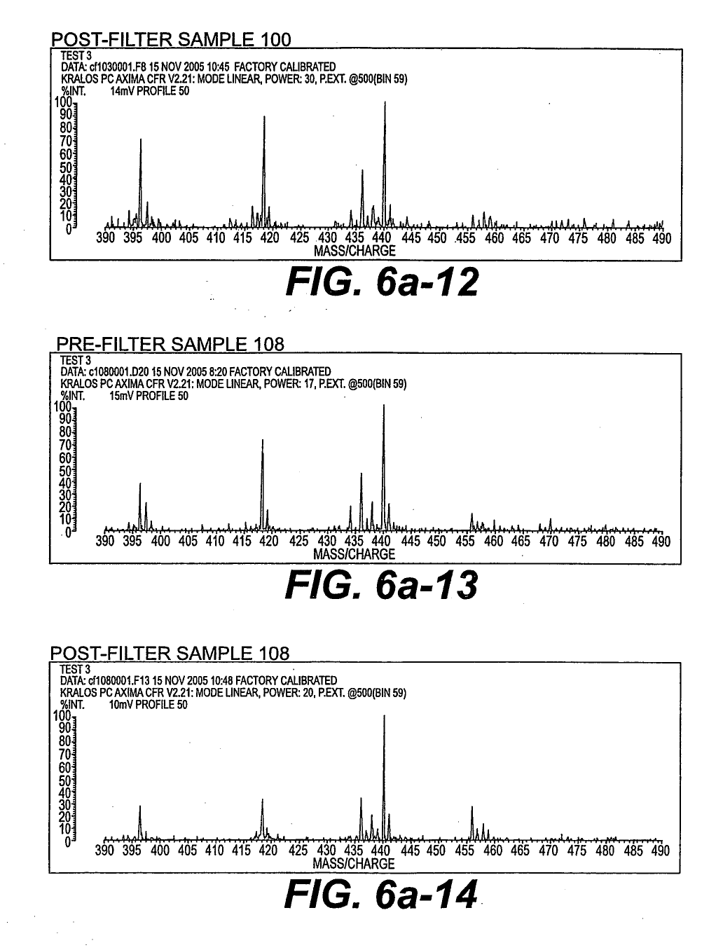

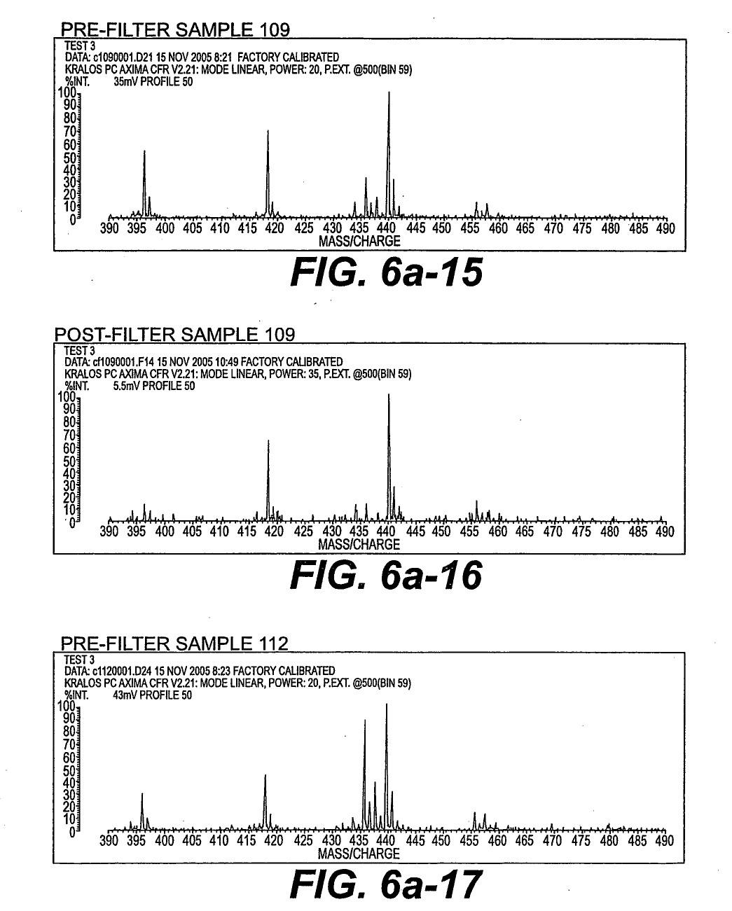

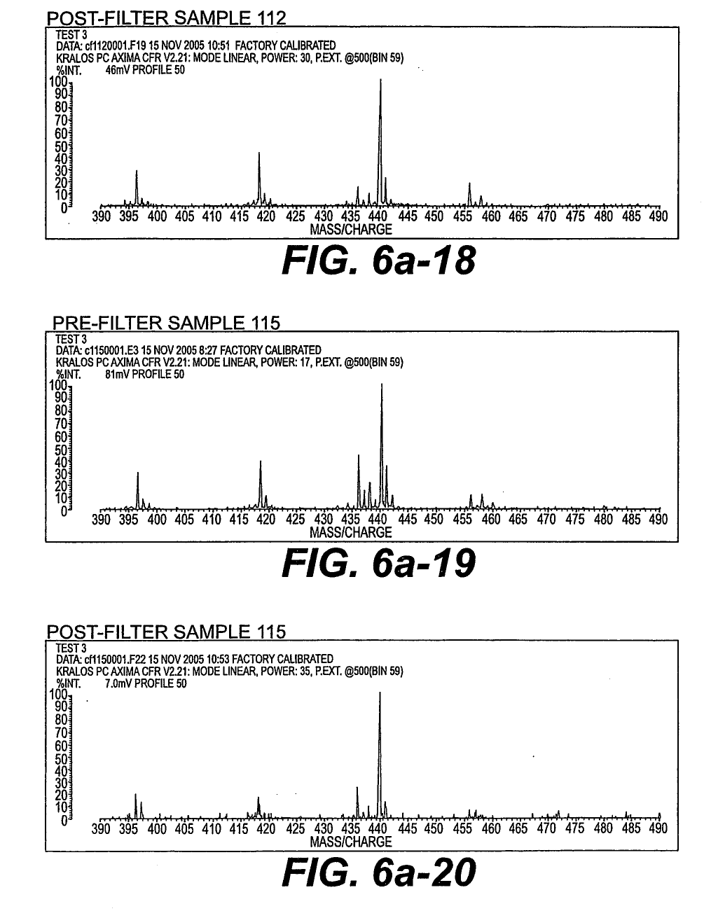

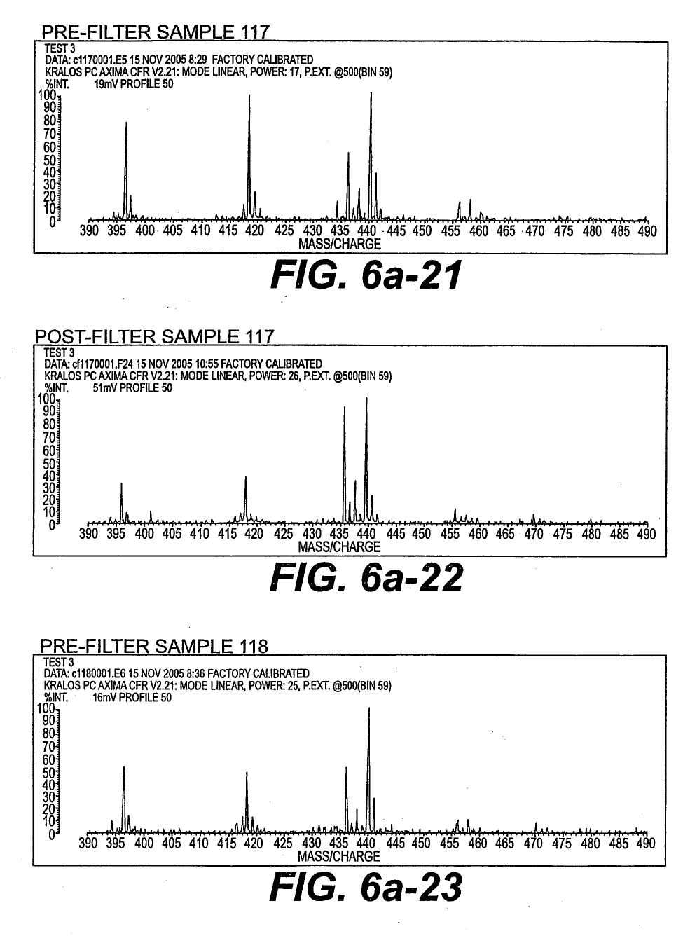

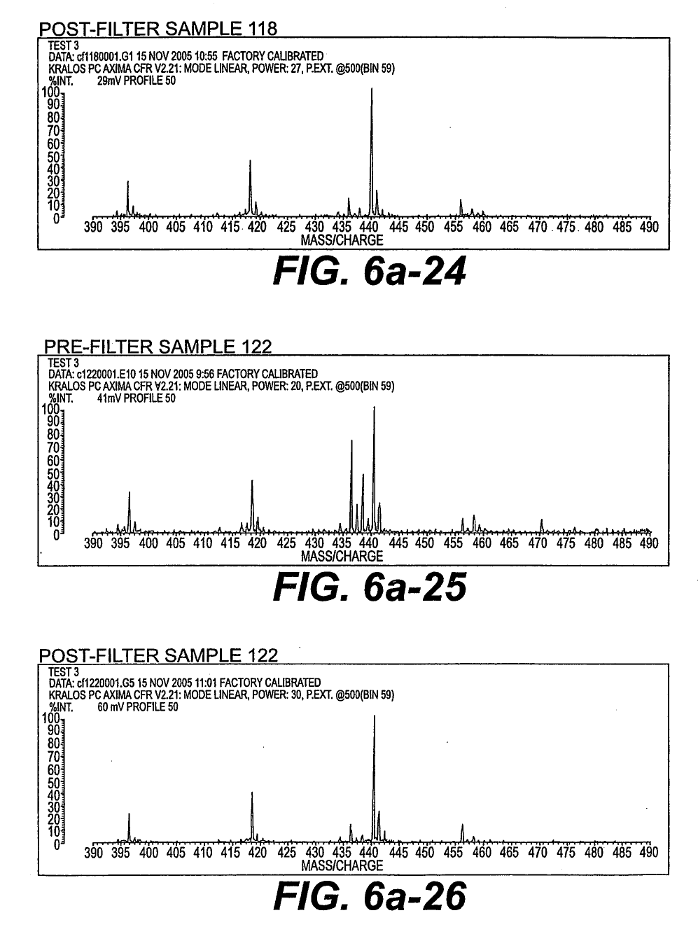

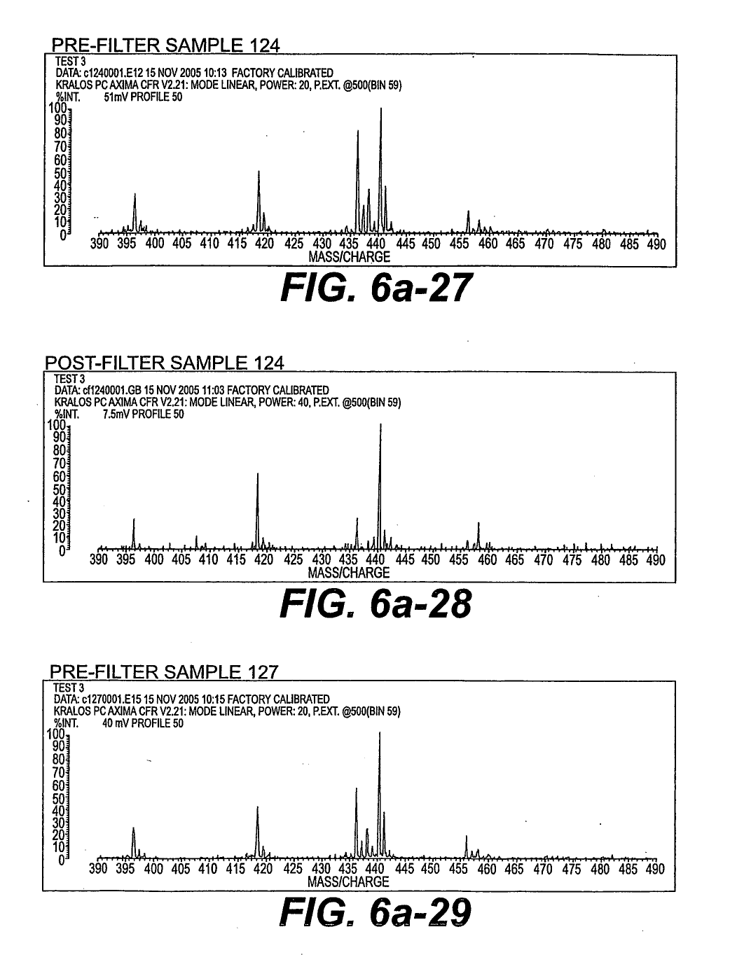

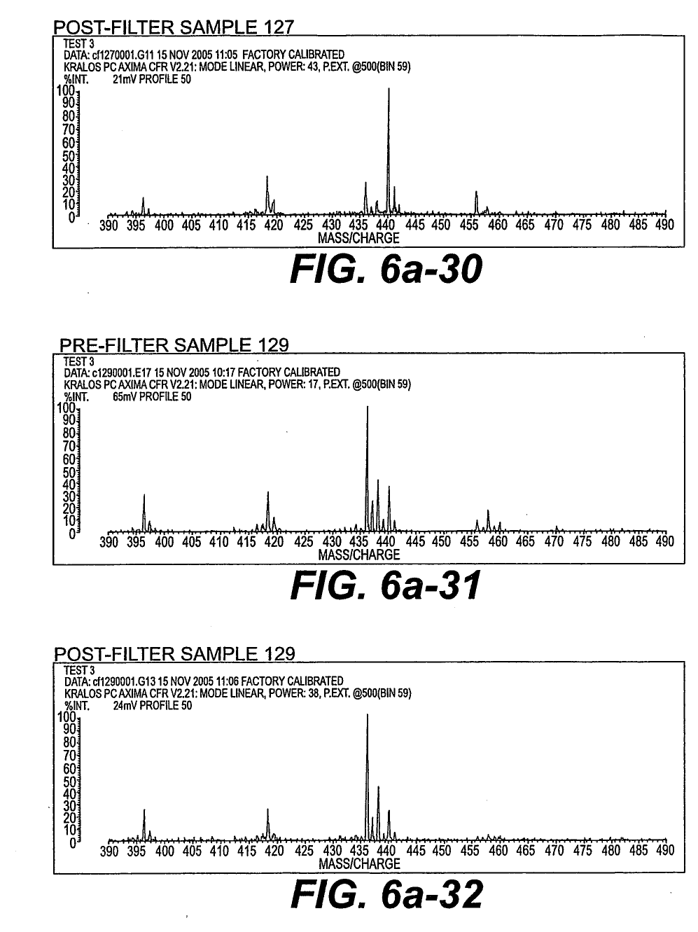

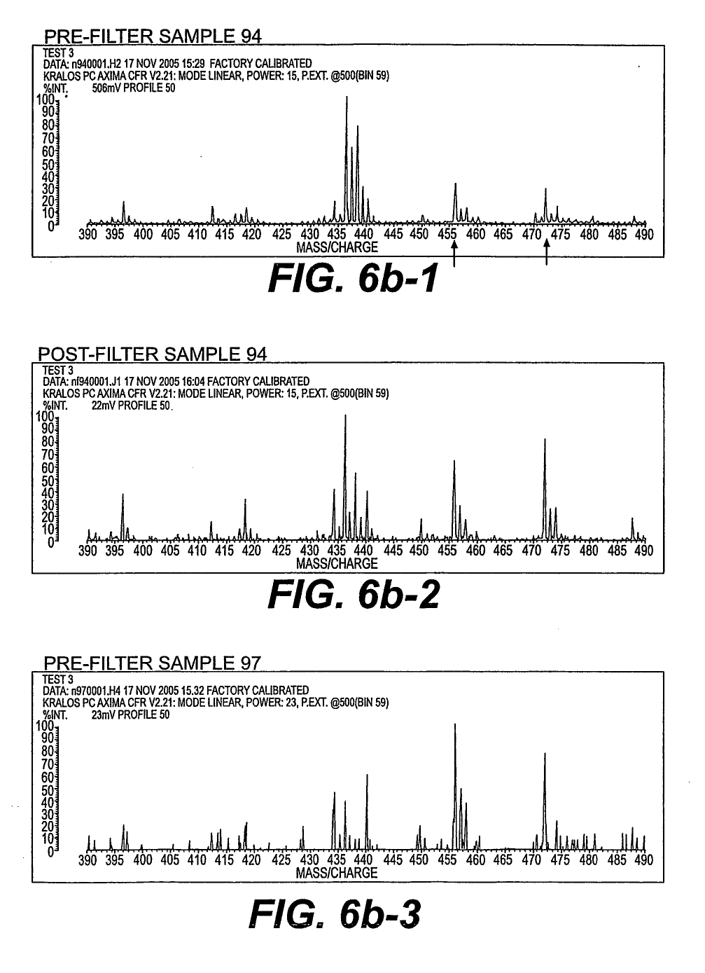

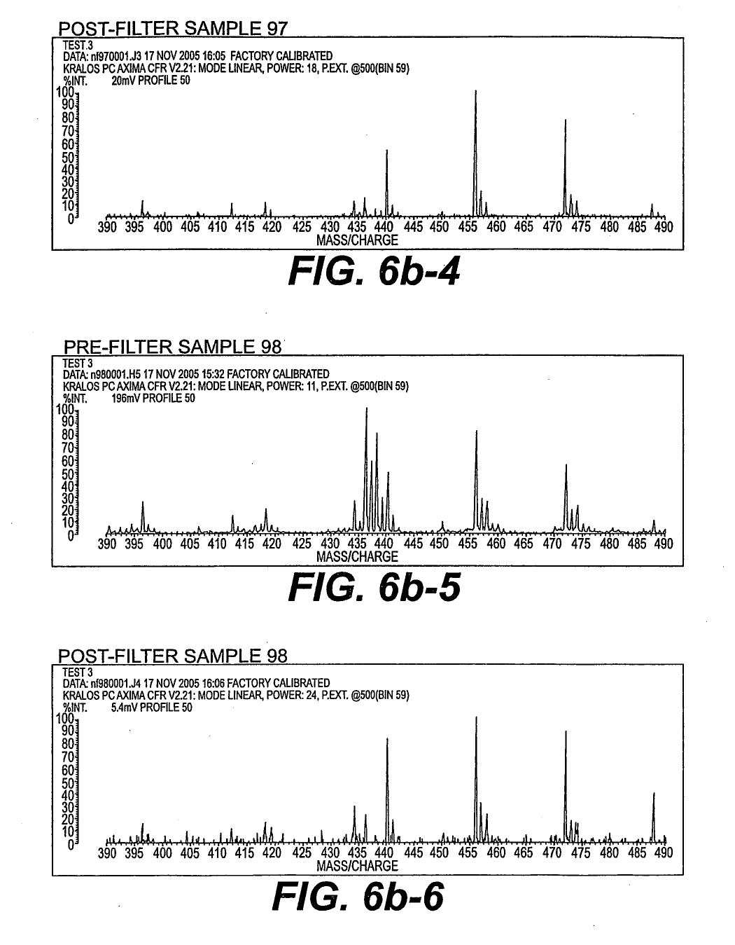

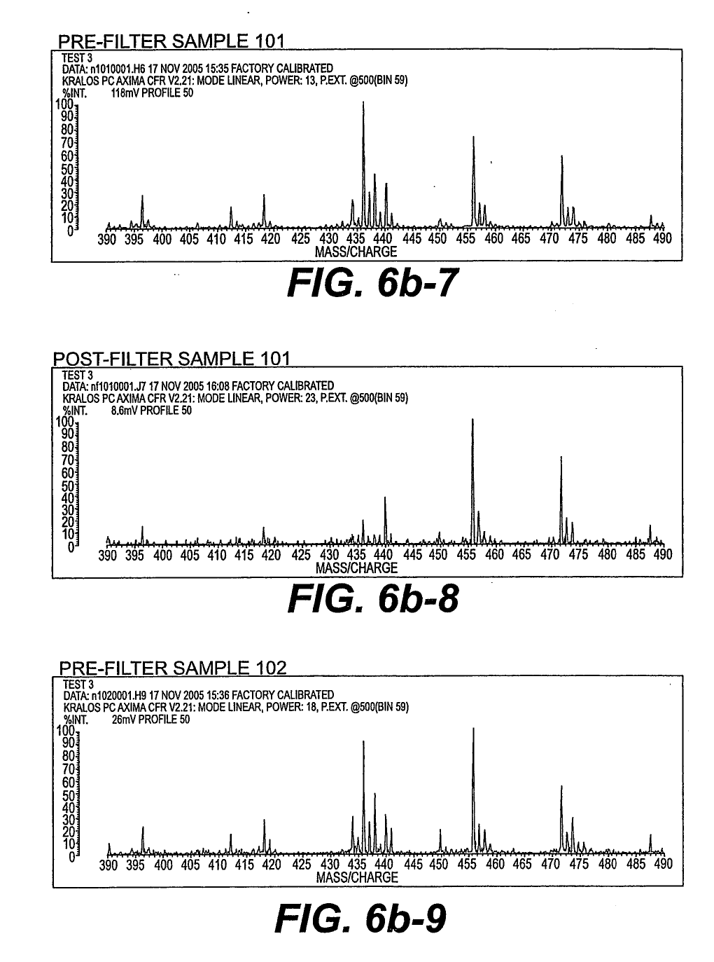

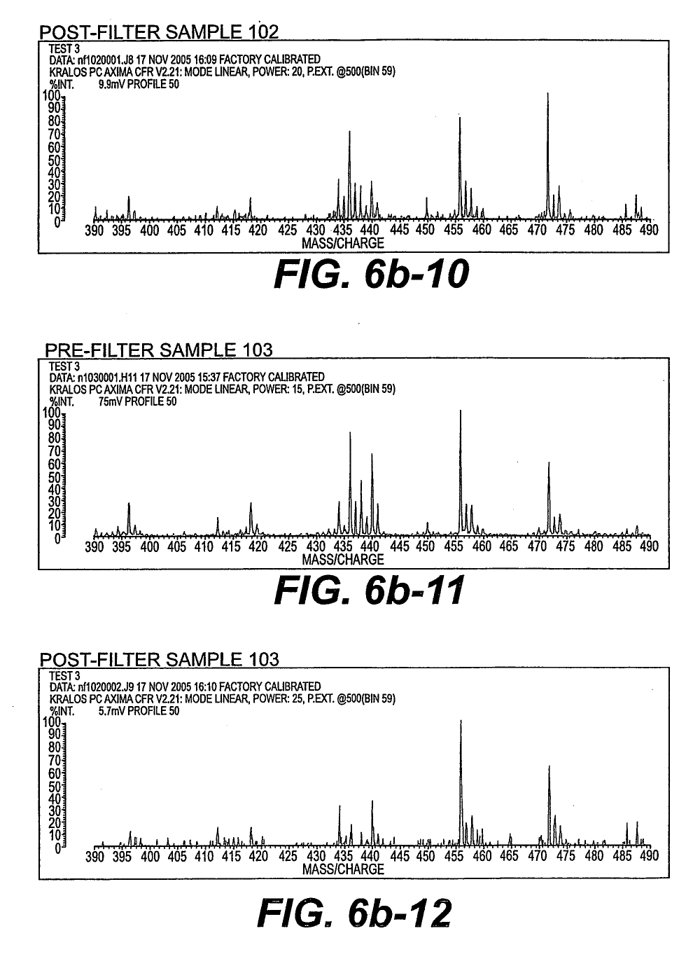

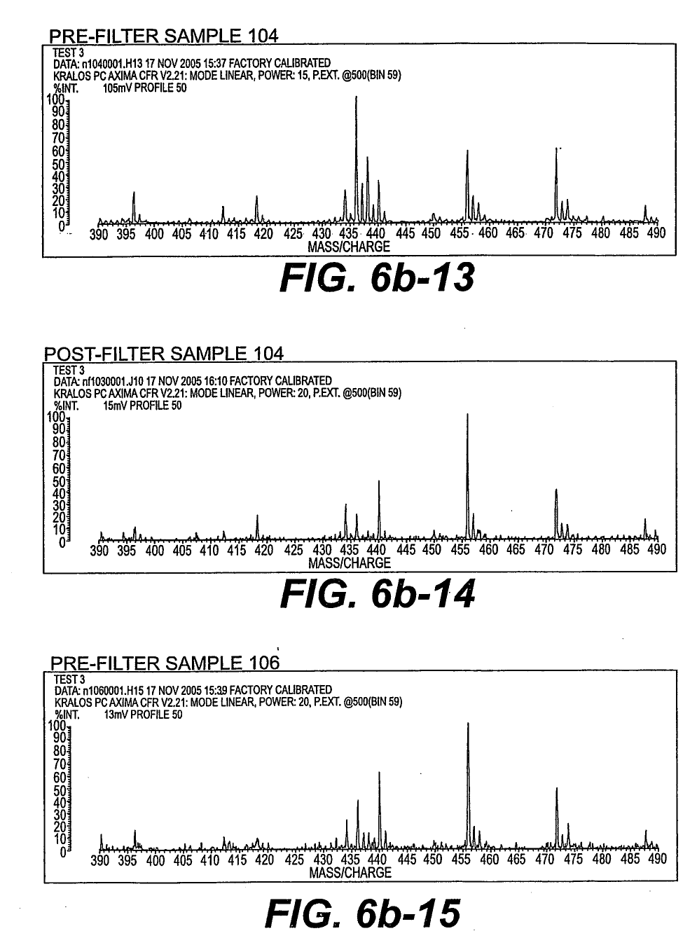

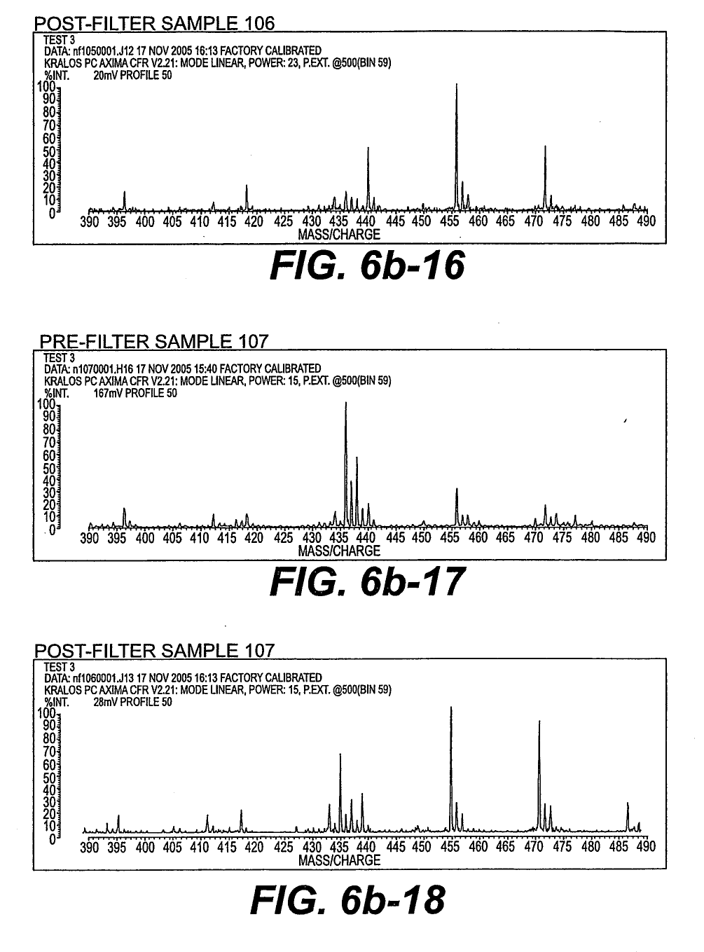

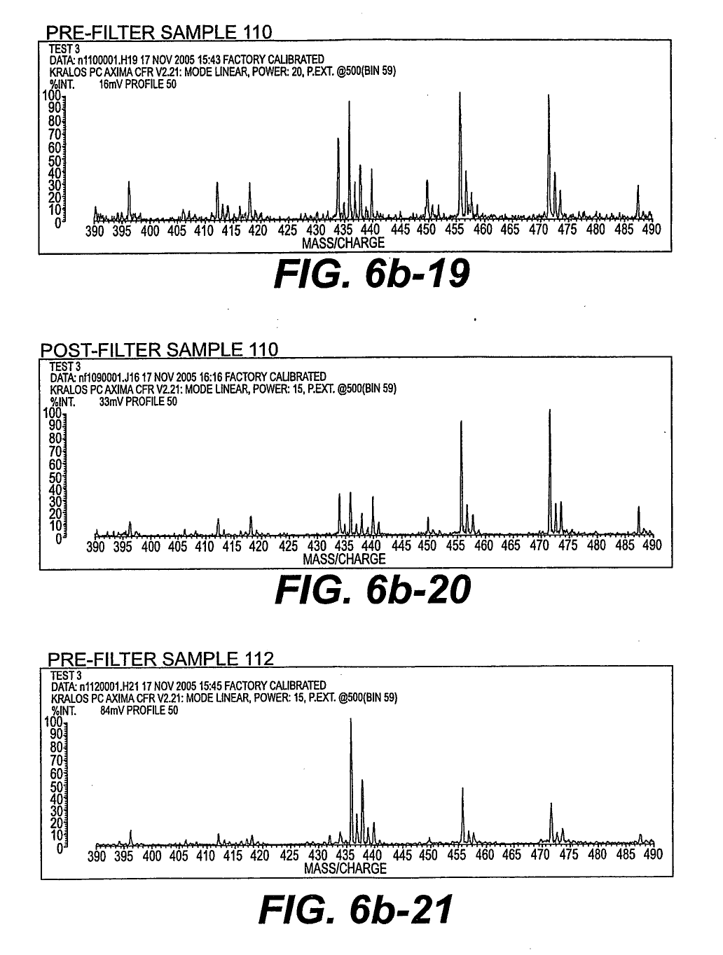

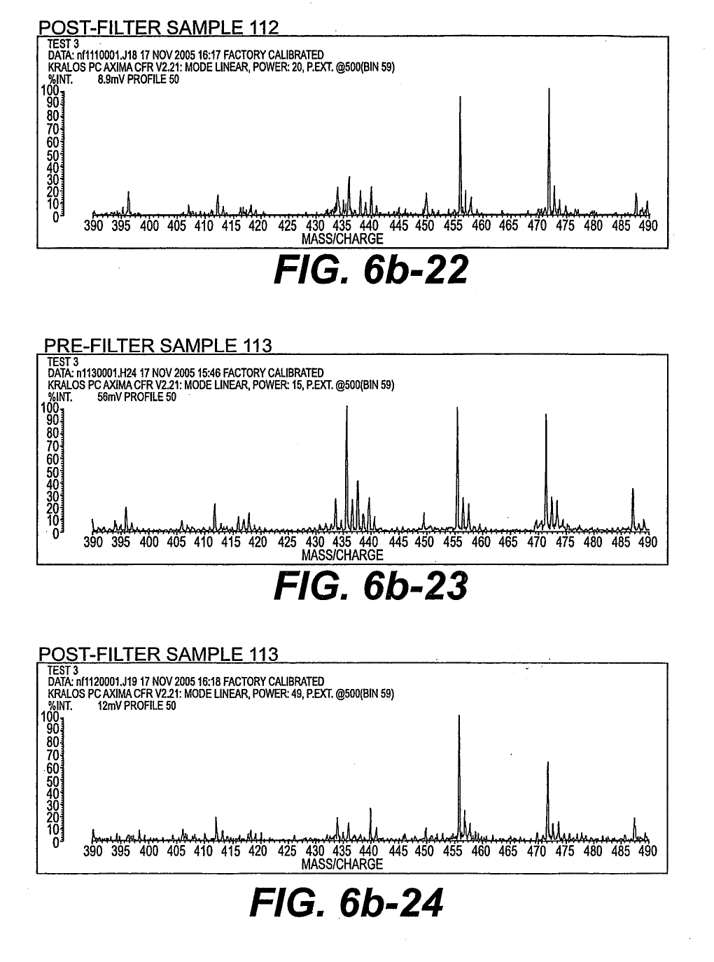

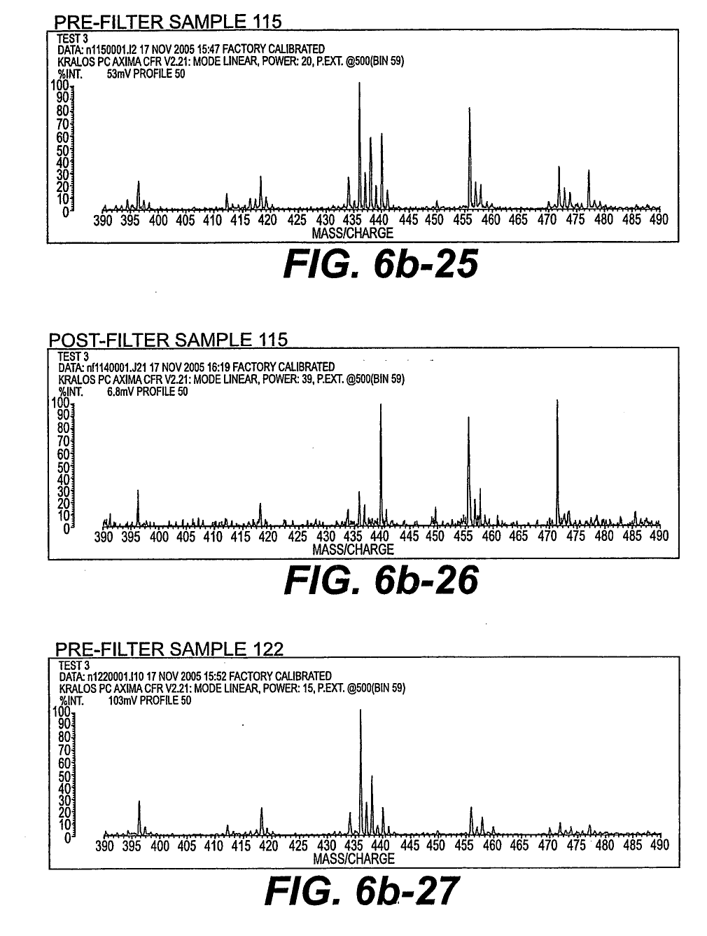

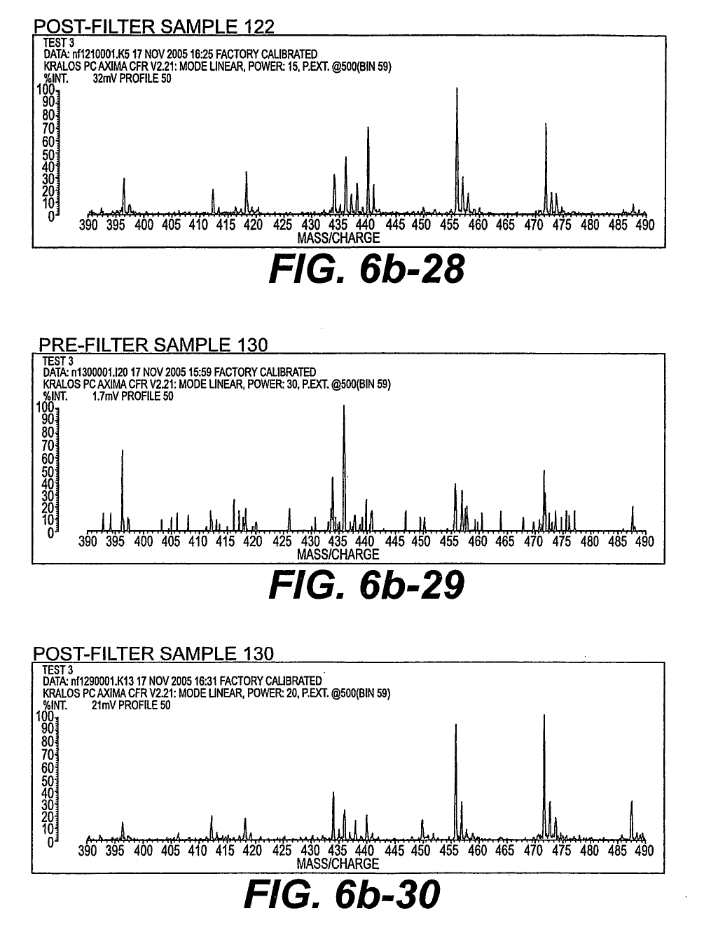

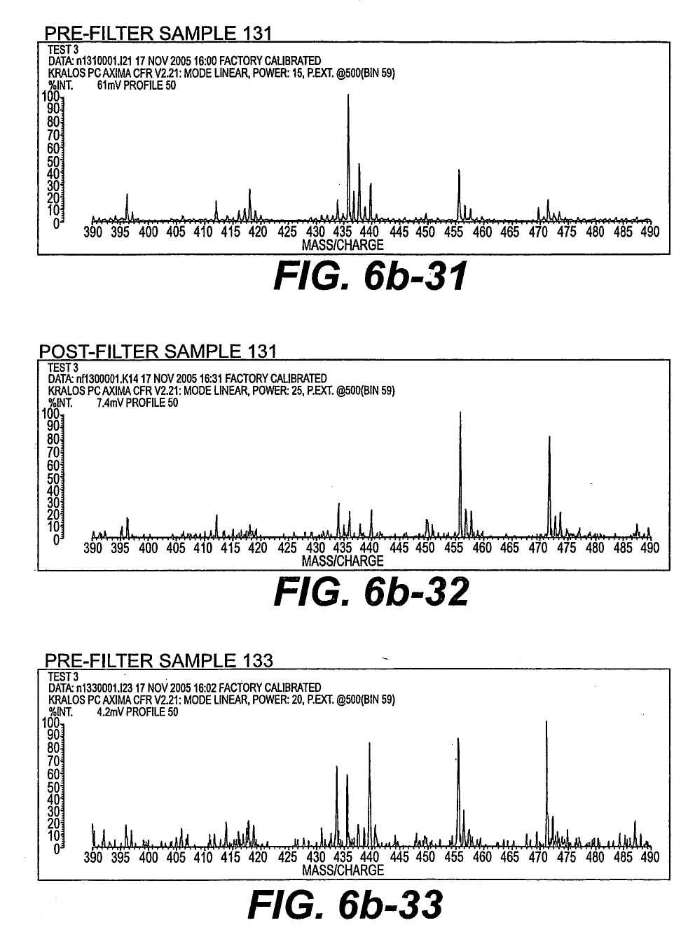

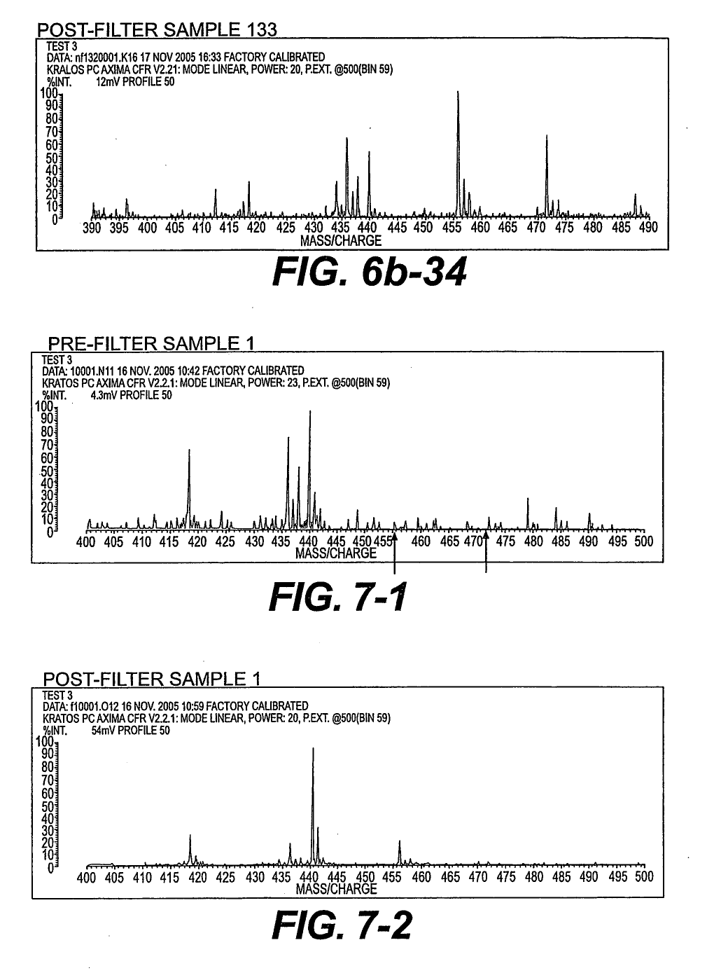

The resulting data, as shown in FIGS. 2 and 6a-6b, show that the use of the filter accentuated the differences between the pre-post filter chromatograms. This enhancement improves the detection of the peaks.

In particular, the data in FIG. 2 indicates that the filtering of the sera resulted in a 10% increase in sensitivity at spot A and a 9% sensitivity increase at spot B Further, specificity was increased by 25% in the case of disease A sera. ( 6/8 (pre-filtering) 75%→ 8/8 (filtering) 100%.

It should be noted that disease A is not meant to be limited to any particular disease. The present invention is applicable to any disease that may show a difference in mass chromatograms compared to those of normal patients. Exemplary diseases include cancer.

### EXAMPLE 3

Method and Apparatus

Example embodiments are now described with respect to FIG. 1 and FIG. 5. FIG. 1 is a flow diagram that illustrates an overview of one embodiment of a method of predicting response (or lack of response) to a particular disease treatment.

Generating Data from Un-Filtered Serum Samples

MALDI-TOF-MS was used to generate a spectra sample data set representing distinct protein/peptide patterns in serum.

The sera were prepared for evaluation by the mass spectrometer by making a matrix of serum samples. The mass spectrometer matrix contained saturated alpha-cyano-4-hydroxycinnamic acid in 50% acetonitrile-0.05% trifluoroacetic acid (TFA). The sera were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Generating Data from Filtered Serum Samples

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1). The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spread out, filling the inner square of filter. The negative air flow was applied by pumping the air pump manually. The dropped sera solutions from the filter to the micro tube were collected and to transferred to the new tube. The filtered serum was further diluted 1:100 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Results

Analysis of the chromatograms show that a peak was present at a particular spot in a substantial number of the non-responders that is not present in the responders. These results can be seen in FIG. 5.

The above example is not meant to limit the disclosed invention to treatments for any particular disease. Exemplary treatments include chemotherapies for treatment of cancer.

### EXAMPLE 4

Method and Apparatus

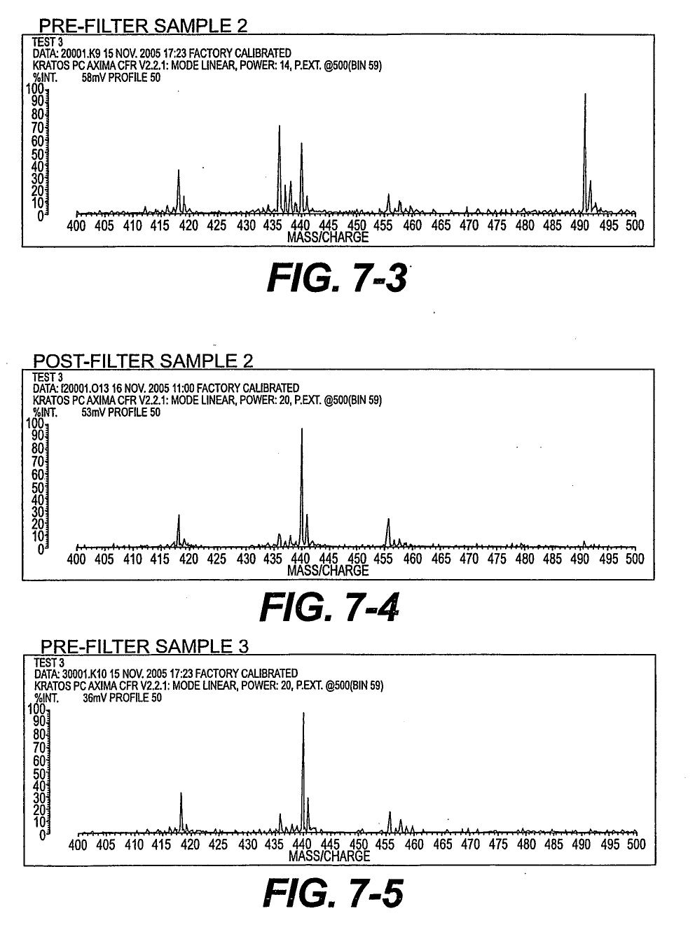

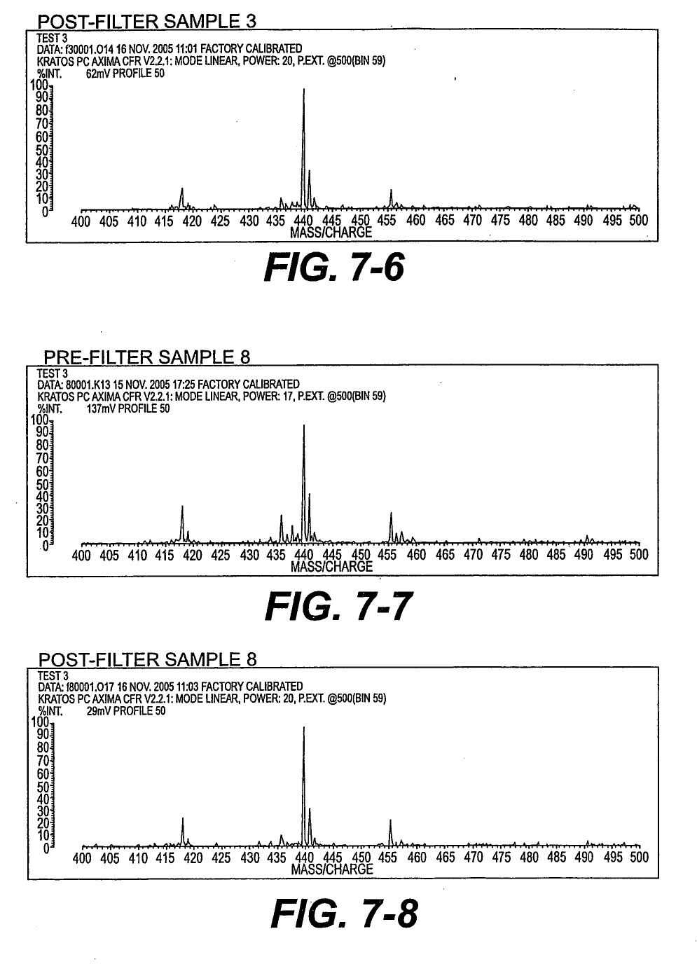

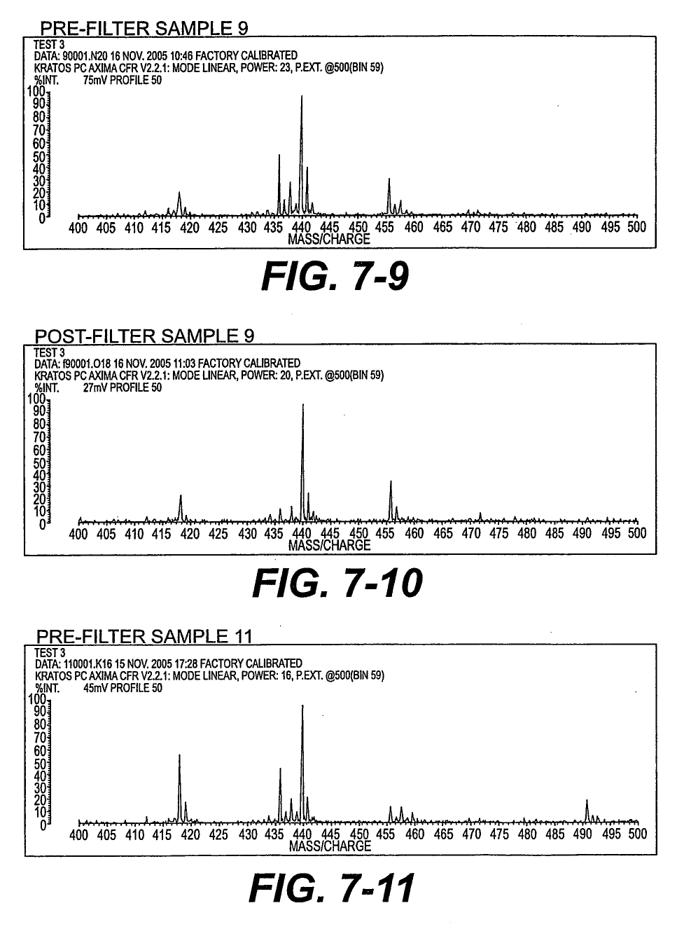

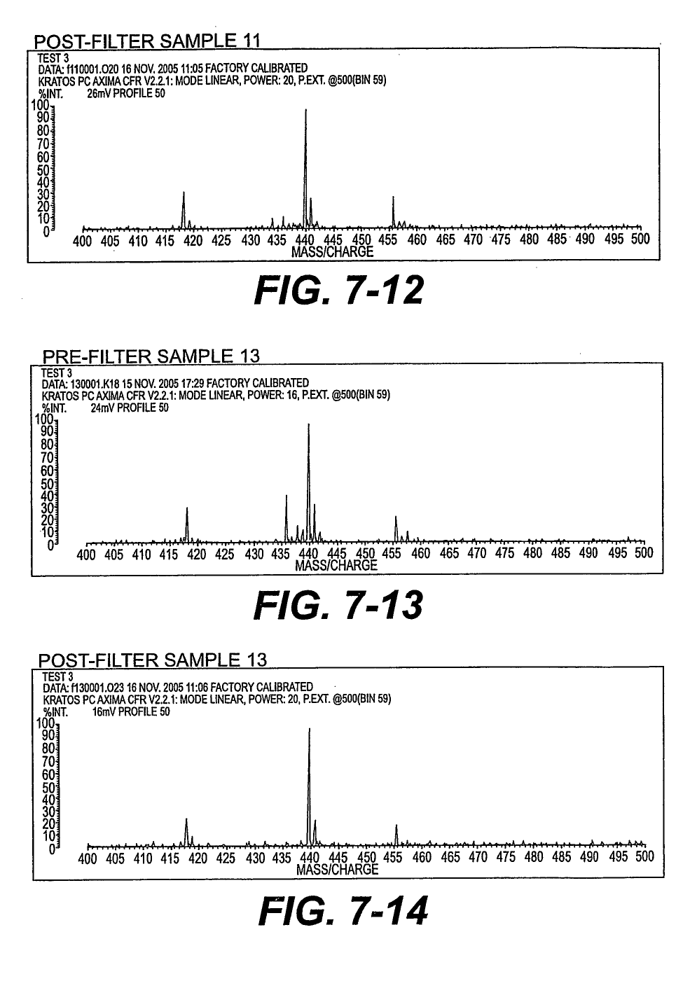

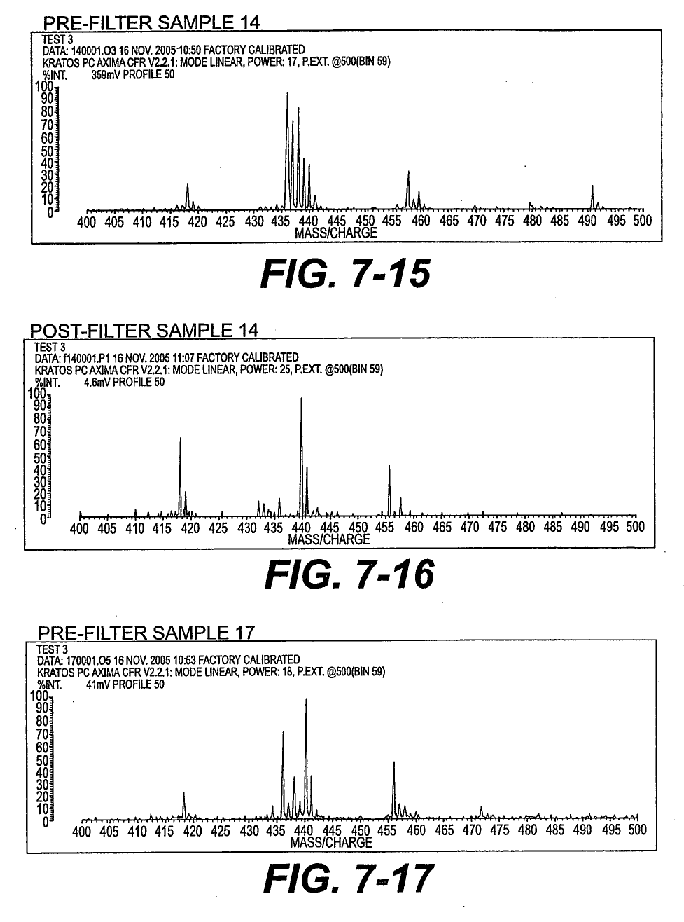

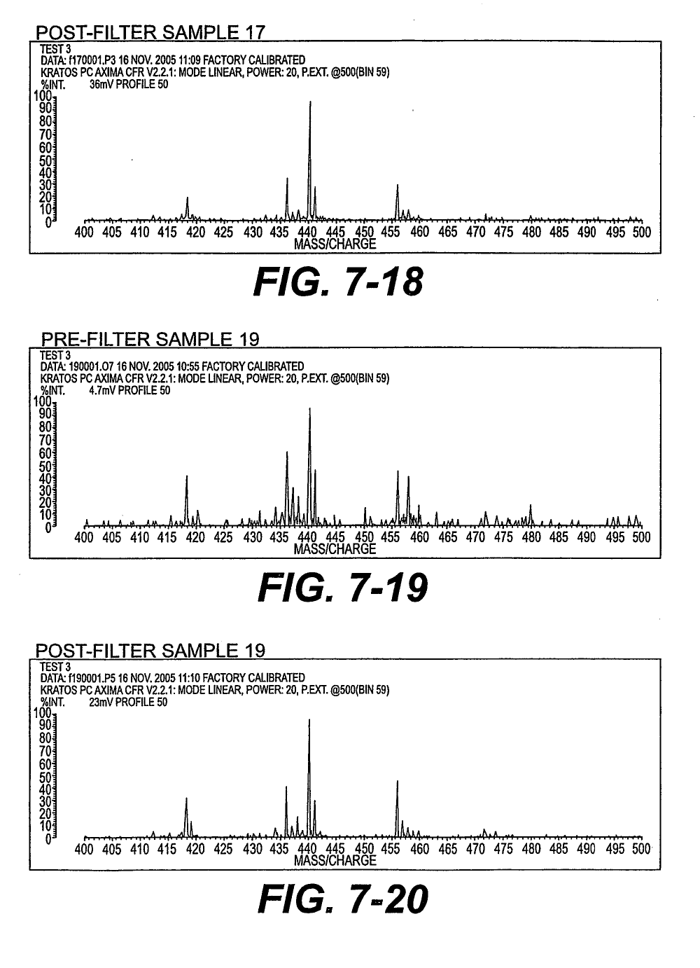

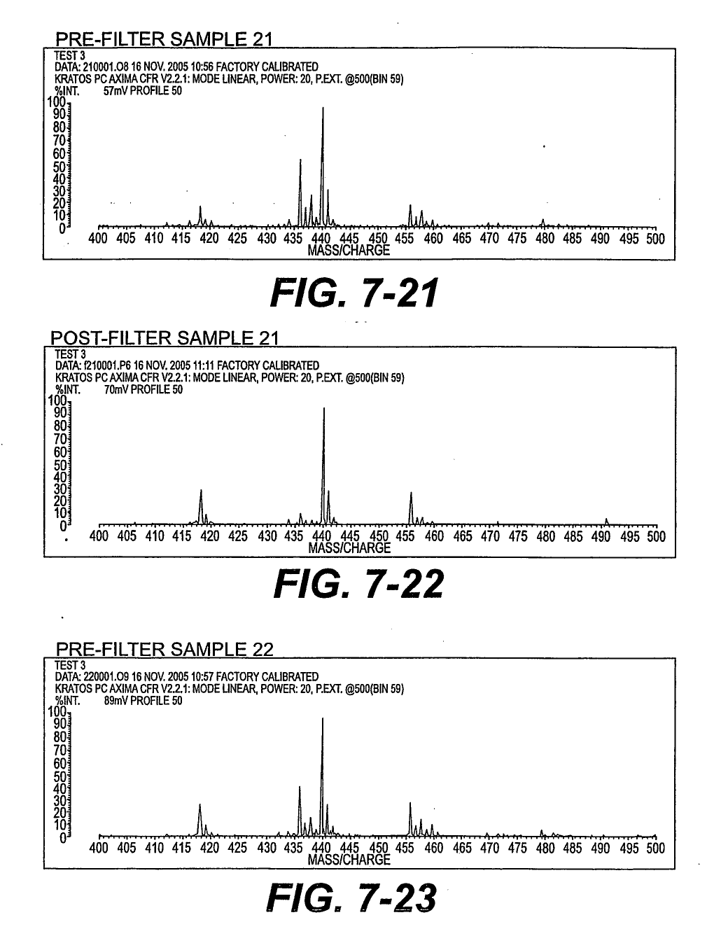

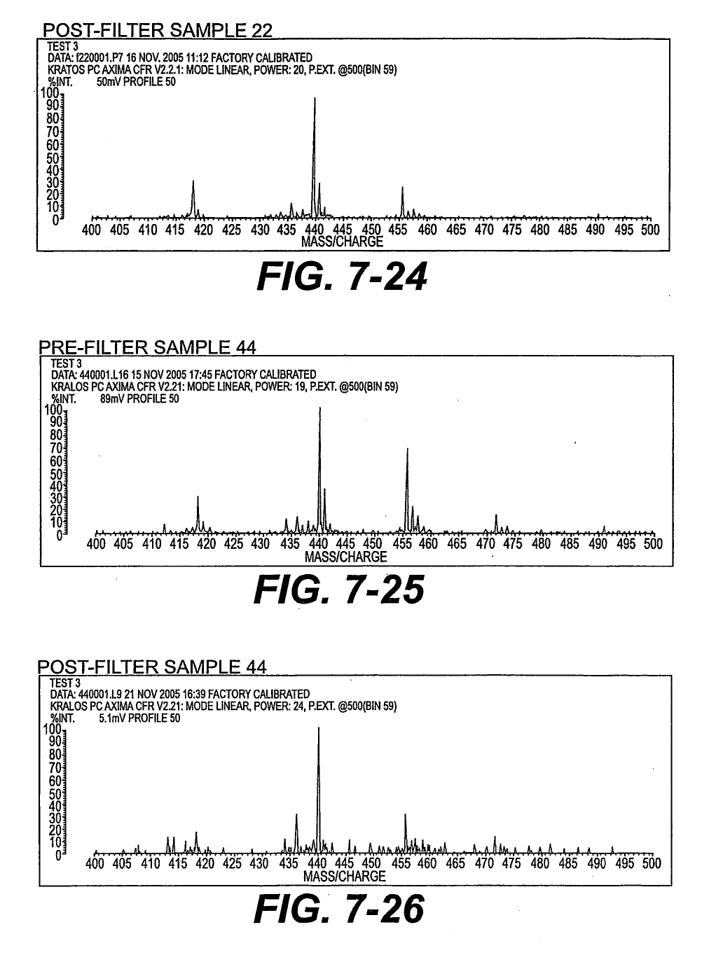

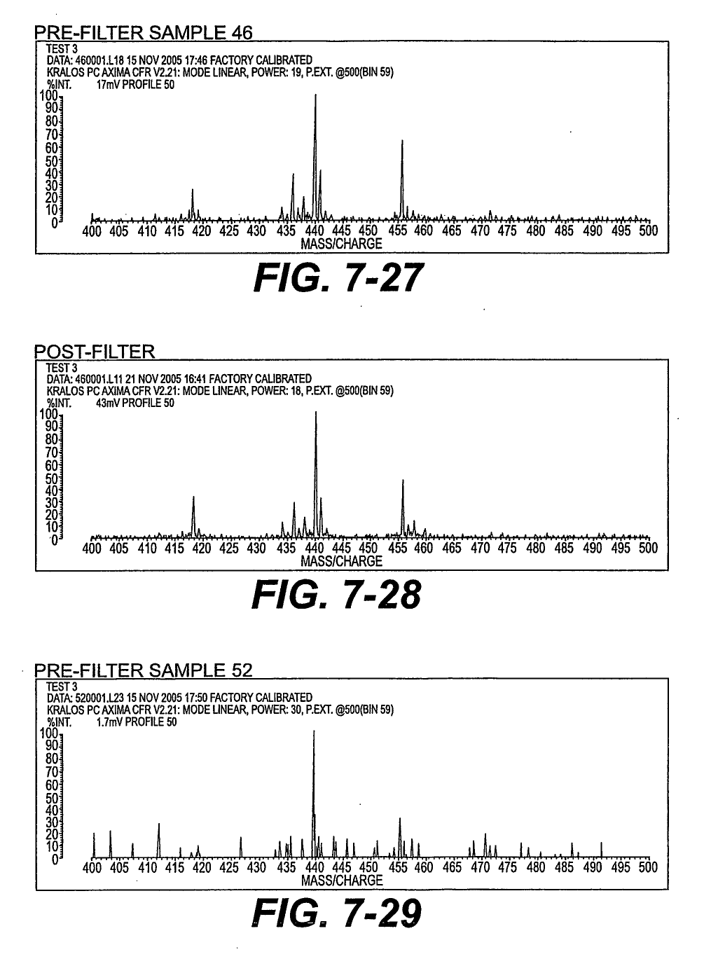

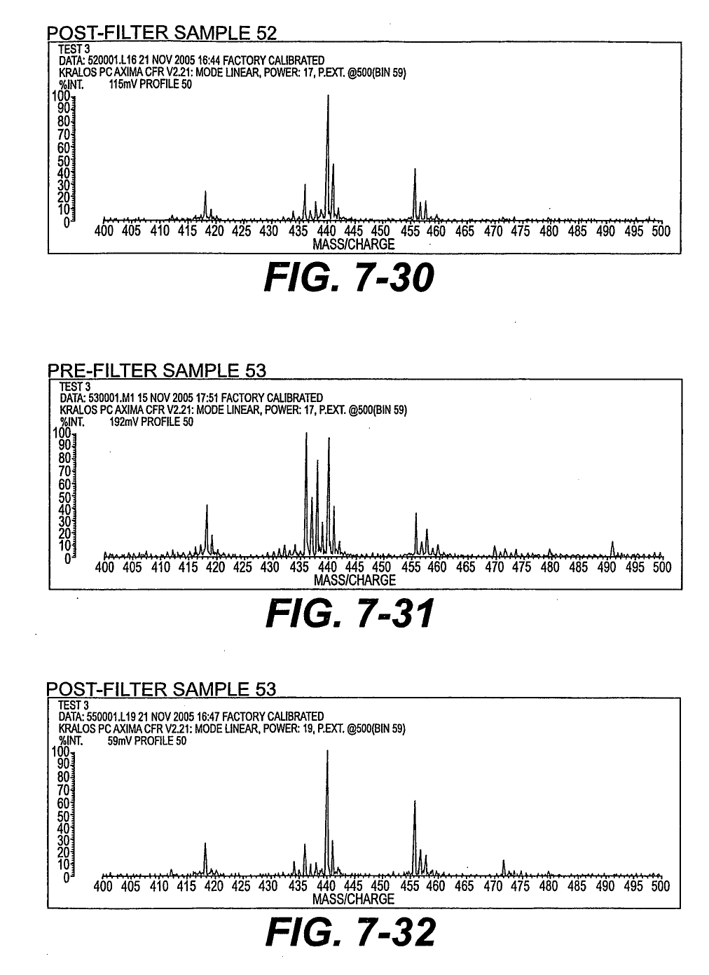

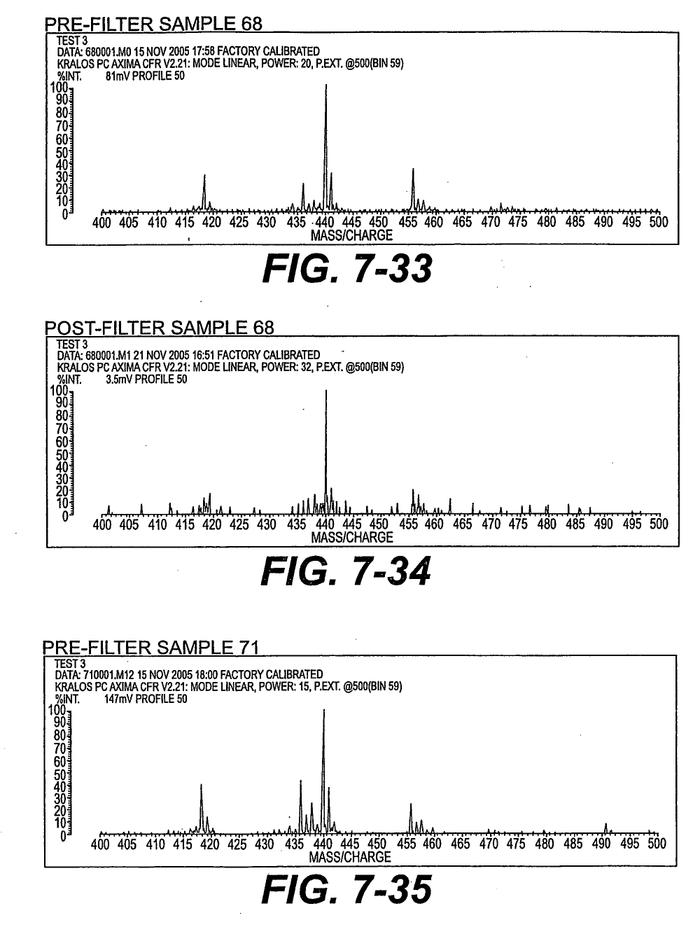

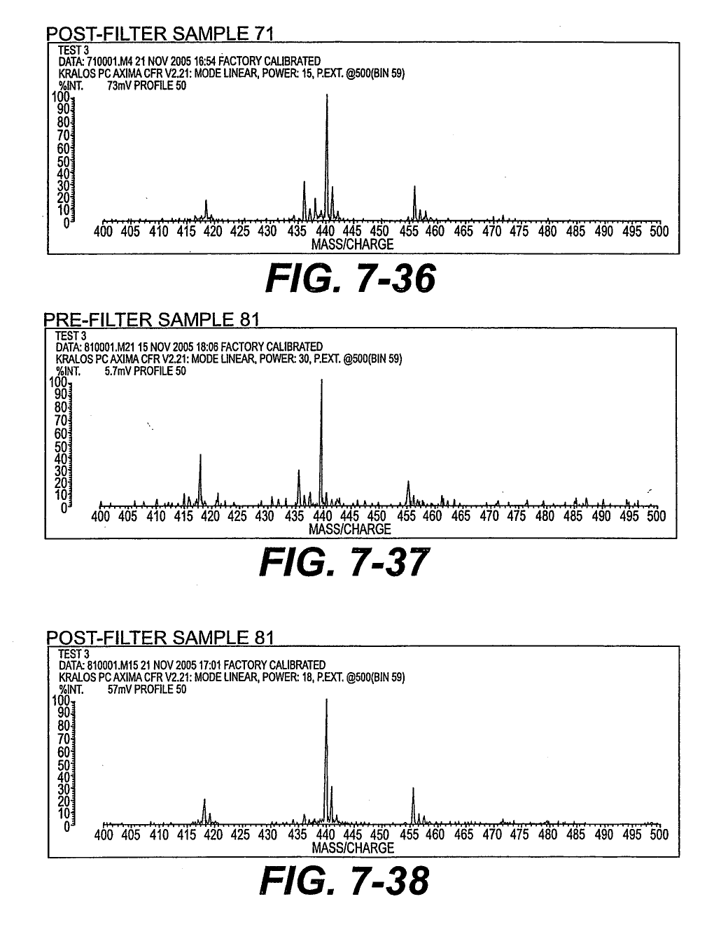

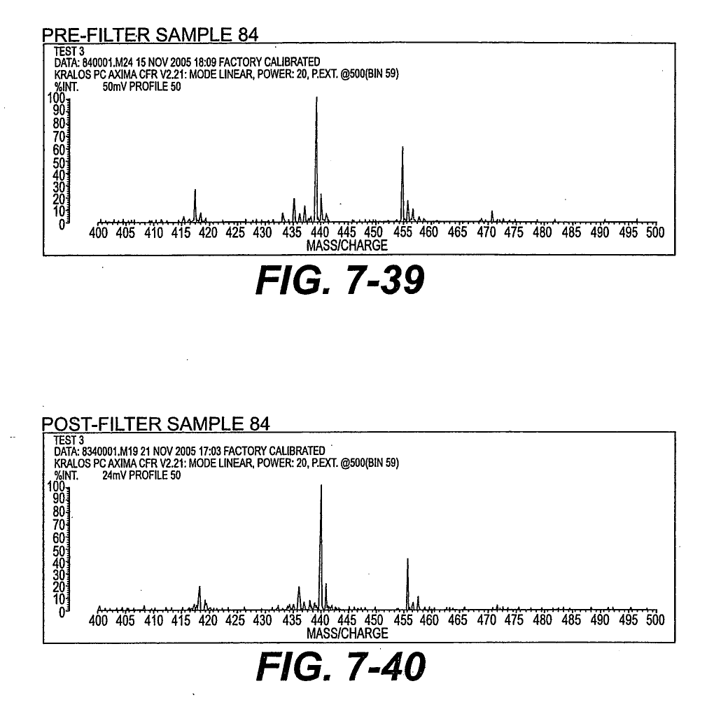

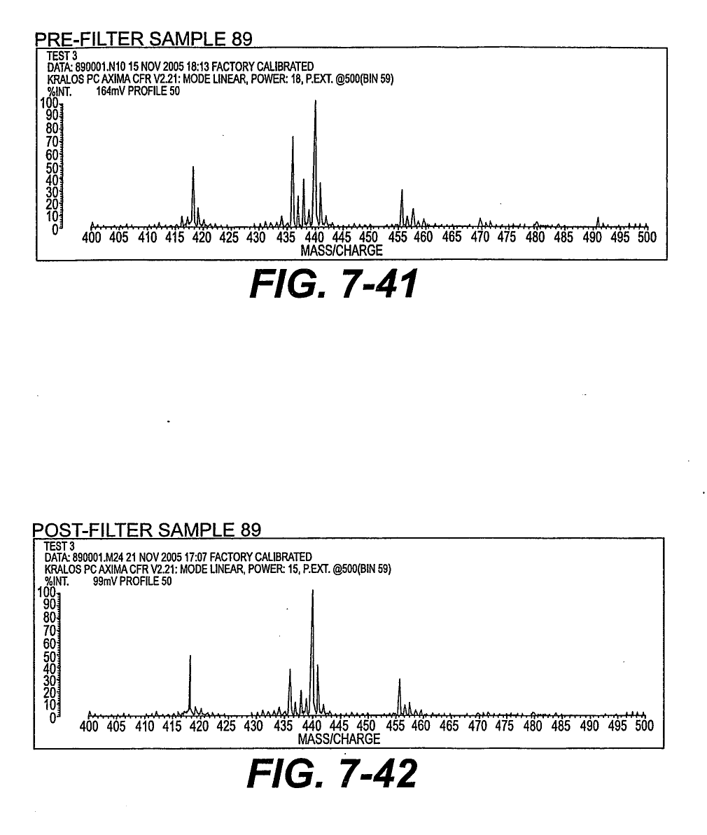

Example embodiments are now described with respect to FIG. 1 and FIG. 7. FIG. 1 is a flow diagram that illustrates an overview of one embodiment of a method for generating a disease B-screening.

Generating Data from Un-Filtered Serum Samples

MALDI-TOF-MS was used to generate a spectra sample data set representing distinct protein/peptide patterns in serum. In one clinical investigation, fluid either from patients with disease A or healthy controls were obtained before surgical procedures. All final diagnoses were confirmed by histopathology and all controls were at high risk for disease B, but without evidence of disease B based on clinical presentation and CT scan examination.

The fluids were prepared for evaluation by the mass spectrometer by making a matrix of serum samples. The mass spectrometer matrix contained saturated alpha-cyano-4-hydroxycinnamic acid in 50% acetonitrile-0.05% trifluoroacetic acid (TFA). The fluids were diluted 1:1000 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Generating Data from Filtered Serum Samples

Before applying the filter, the serum was diluted 1:10 in 0.1% n-Octyl β-D-Glucopyranoside. The micro tubes were cut individually from Micro Amp 8 strip tubes. A hole was made in the bottom part of the micro tube by using a needle (Becton Dickinson 20G1). The micro tube with hole was placed on the metal plate of a gel-pak suction apparatus (air pump) and the hole was adjusted to the same air-flow direction of the air pump. The filter was placed on the top of the micro tube. 20 μl of serum was loaded on the upper part of filter. The serum solution spreads out, filling the inner square of filter. The negative air flow was applied by pumping the air pump manually. The dropped sera solutions from the filter to the micro tube were collected and transferred to the new tube. The filtered serum was further diluted 1:100 in 0.1% n-Octyl β-D-Glucopyranoside. 0.5 μL of the matrix was placed on each defined area of a sample plate with 384 defined areas and 0.5 μL serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Results

The use of the filter accentuated the differences between the pre-filter and post-filter chromatograms. This enhancement improves the detection of the peaks. These results can be seen in FIG. 7.

It should be noted that disease B is not meant to be limited to any particular disease. The present invention is applicable to any disease that may show a difference in mass chromatograms compared to those of normal patients. Exemplary diseases include cancer.

### EXAMPLE 5

Method and Apparatus

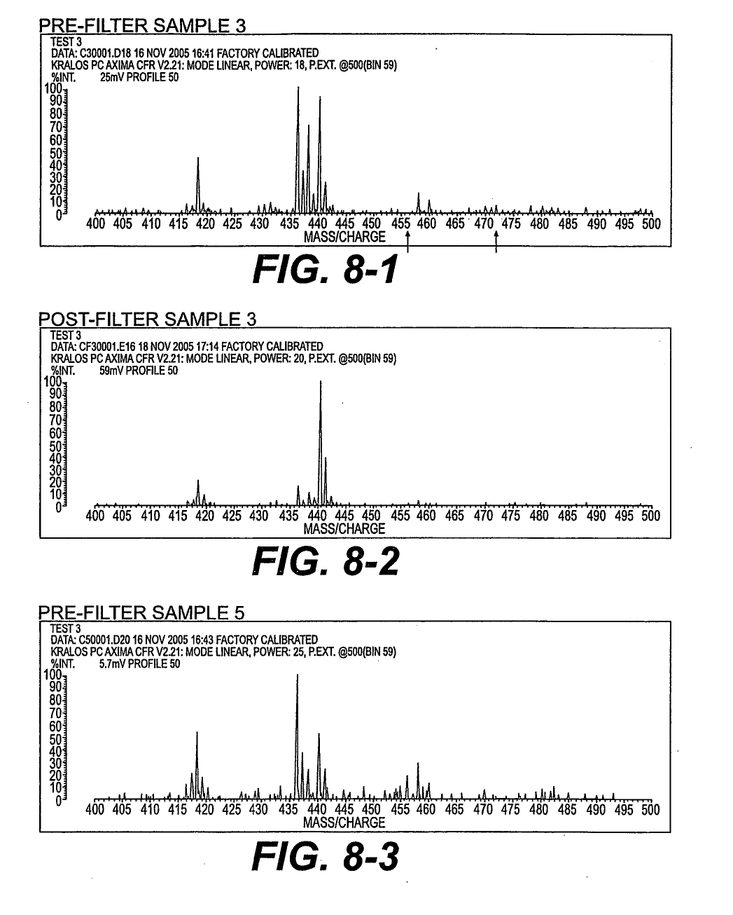

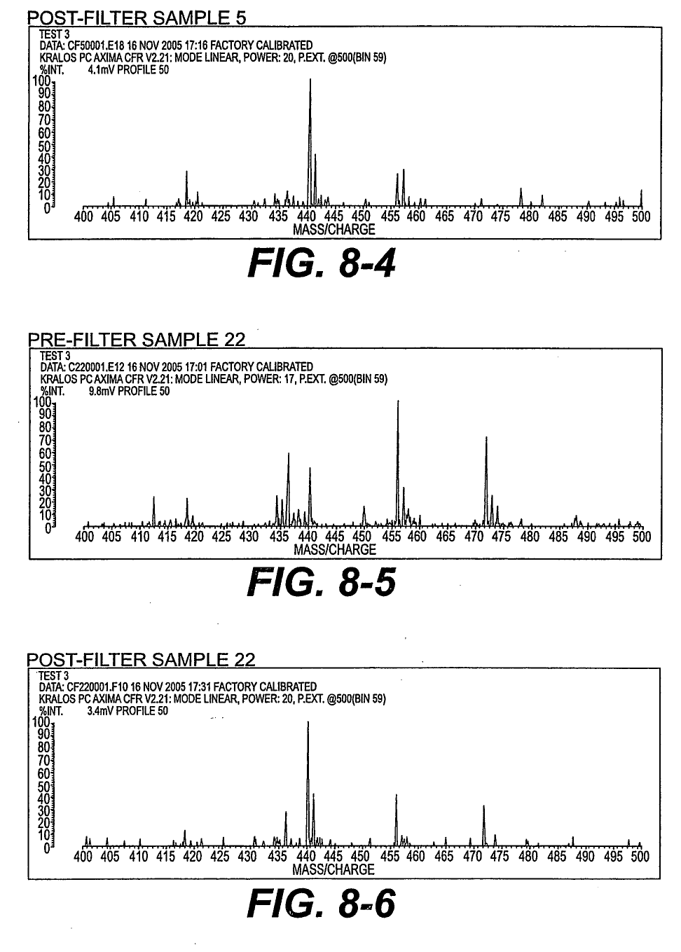

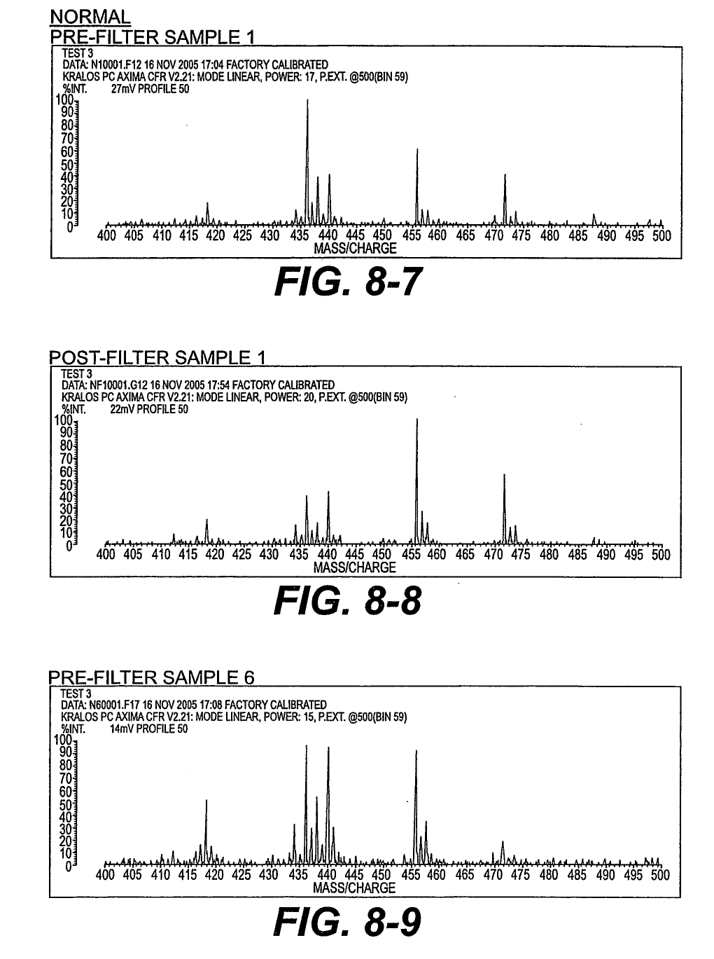

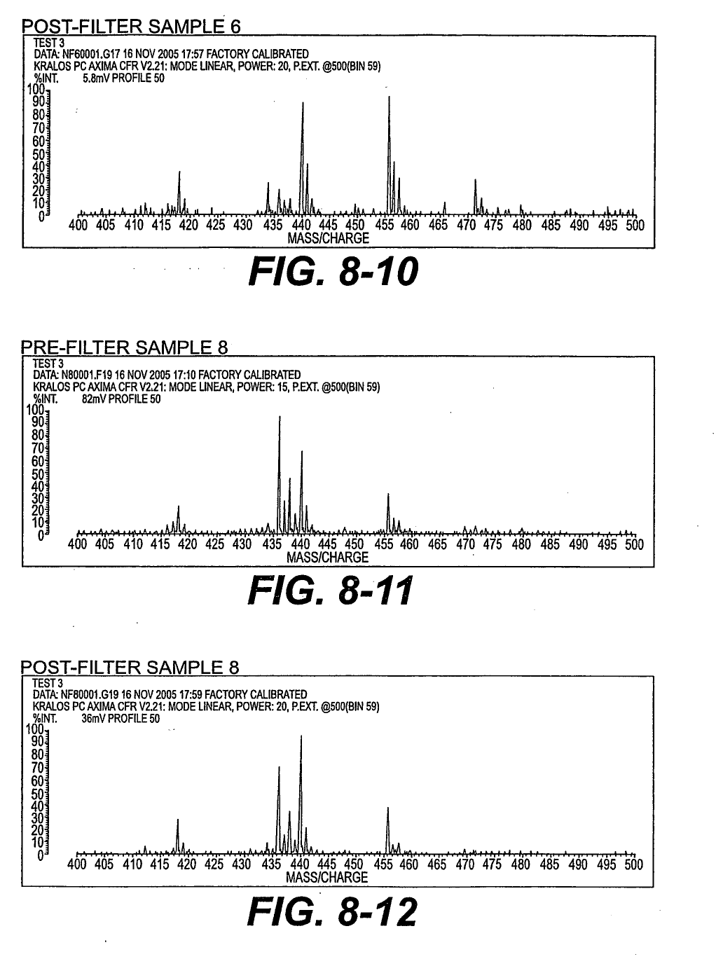

Example embodiments are now described with respect to FIG. 1 and FIG. 8. FIG. 1 is a flow diagram that illustrates an overview of one embodiment of a method for generating a disease C-screening.

Generating Data from Un-Filtered Urine Samples

MALDI-TOF-MS was used to generate a spectra sample data set representing distinct protein/peptide patterns in urine. In one clinical investigation, urine either from patients with disease C or healthy controls were obtained before surgical procedures. All final diagnoses were confirmed by histopathology and all controls were at high risk for disease C, but without evidence of disease C based on clinical presentation and CT scan examination.

The fluids were prepared for evaluation by the mass spectrometer by making a matrix of urine samples. The mass spectrometer matrix contained saturated alpha-cyano-4-hydroxycinnamic acid in 50% acetonitrile-0.05% trifluoroacetic acid (TFA). The fluids were diluted 1:1000 in 0.1% n-Octyl β-D-Glucopyranoside. 0.5 μL of the matrix was placed on each defined area of a sample plate with 384 defined areas and 0.5 μL urine from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Generating Data from Filtered Urine Samples

Before applying the filter, the urine was diluted 1:10 in 0.1% n-Octyl β-D-Glucopyranoside. The micro tubes were cut individually from Micro Amp 8 strip tubes. A hole was made in the bottom part of the micro tube by using needle (Becton Dickinson 20G1). The micro tube with hole was placed on the metal plate of a gel-pak suction apparatus (air pump) and the hole was adjusted to the same air-flow direction of the air pump. The filter was placed on the top of the micro tube. 20 μl of urine was loaded on the upper part of filter. The urine solution spreads out, filling the inner square of filter. The negative air flow was applied by pumping the air pump manually. The dropped urine solutions from the filter to the micro tube were collected and to transferred to the new tube. The filtered urine was further diluted 1:100 in 0.1% n-Octyl β-D-Glucopyranoside. 0.5 μL of the matrix was placed on each defined area of a sample plate with 384 defined areas and 0.5 μL urine from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set to the following specifications: tuner mode, linear; mass range, 0 to about 5,000; laser power, 90; profile, 100; shots per spot, 5. The output of the mass spectrometer was stored in computer storage in the form of a sample data set.

Results

The use of the filter accentuated the differences between the pre-post filter chromatograms. This enhancement improves the detection of the peaks. These results can be seen in FIG. 8.

It should be noted that disease C is not meant to be limited to any particular disease. The present invention is applicable to any disease that may show a difference in mass chromatograms compared to those of normal patients. Exemplary diseases include cancer.

While the invention has been described with reference to certain exemplary embodiments thereof, those skilled in the art may make various modifications to the described embodiments of the invention without departing from the scope of the invention. The terms and descriptions used herein are set forth by way of illustration only and are not meant as limitations. In particular, although the present invention has been described by way of examples, a variety of compositions and methods would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

1. A method for determining the probability of disease, comprising:
filtering a biological fluid through a hole array filter;
generating mass spectra data from the filtered biological fluid; and
comparing the mass spectra data with a database.

2. The method of claim 1, wherein the disease is lung cancer.

3. The method of claim 1, wherein the disease is bladder cancer.

4. The method of claim 1, wherein the mass spectra data is taken by MALDI-TOF-MS.

5. The method of claim 1, wherein the biological fluid is serum.

6. The method of claim 1, wherein the biological fluid is urine.

7. The method of claim 1, wherein the hole array filter comprises holes with a diameter of at least about 1 to 10 μm.

8. The method of claim 1, wherein the hole array filter includes a hole array layer having a thickness of at least about 6 to 10 μm.

9. The method of claim 1, wherein the hole array filter comprises:
a first area with a hole array; and
a second area for maintaining filter rigidity, the second area having a thickness greater than the thickness of the first area.

10. The method of claim 1, wherein the hole array filter comprises a first Si layer, an SiO₂ layer and a second Si layer, the first Si layer having a thickness greater than the thickness of the second Si layer.

11-15. (canceled)

16. The method of claim 1, wherein the biological fluid is plasma.
